(19) United States
(12) Patent Application Publication
(10) Pub. No.: US 2013/0267460 A1
(43) Pub. Date: Oct. 10, 2013

(54) Methods of Inhibiting Alphavirus Replication and Treating Alphavirus Infection

(71) Applicants: Diane E. Griffin, Hunt Valley, MD (US); Michael Ayars, Baltimore, MD (US)

(72) Inventors: Diane E. Griffin, Hunt Valley, MD (US); Michael Ayars, Baltimore, MD (US)

(73) Assignee: The Johns Hopkins University, Baltimore, MD (US)

(21) Appl. No.: 13/823,198

(22) PCT Filed: Sep. 16, 2011
(86) PCT No.: PCT/US11/51980

§ 371 Date: Jun. 10, 2013

Related U.S. Application Data
(60) Provisional application No. 61/383,436, filed on Sep. 16, 2010.

(51) Int. Cl. A61K 38/13; A61K 45/06

(52) U.S. Cl. CPC A61K 38/13; A61K 45/06
USPC 514/3.7; 435/366

(57) ABSTRACT

The invention provides methods of inhibiting alphavirus replication and treating alphavirus infection.

### CROSS-REFERENCE TO RELATED APPLICATIONS

This application claims the benefit of priority to U.S. Provisional Application No. 61/383,436, filed on Sep. 16, 2010, the entire contents of which are expressly incorporated herein by reference.

### STATEMENT OF RIGHTS

This invention was made with government support under Grant NIH RO 1 NS018596. The U.S. government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

Viruses of the genus Alphaviridae belong to the group IV Togaviridae family of viruses based on the well-known Baltimore taxonomic classification of viruses. Of the roughly 30 known alphaviruses, at least one third cause significant diseases in humans and animals worldwide, which manifest with such debilitating symptoms as encephalitis, arthritis, rashes, fevers, headache, nausea, myalgia, arthralgia (joint pain), arthropathy (diseases of the joint), chills, diarrhea, vomiting, lymphadenitis, malaise, and muscle soreness. In particular, Sindbis, Semliki Forest, O'nyong'nyong, Chikungunya, Mayaro, Ross River, Barmah Forest, Eastern Equine Encephalitis, Western Equine Encephalitis, and Venezuelan Equine Encephalitis viruses are medically relevant alphaviruses that generally infect human populations via insect vectors (e.g., mosquitoes) and can cause fatal encephalitis if alphaviral infection reaches the central nervous system (CNS). Neurotropic alphaviruses are also important members of the growing list of emerging or resurging public health threats (Gubler (2002) Arch. Med. Res. 33:330-42) and are listed as CDC and NIAID category B bioterrorism agents due in part to numerous characteristics that make them potential biological weapons: (i) high clinical morbidity and mortality; (ii) potential for aerosol transmission; (iii) lack of effective countermeasures for disease prevention or control; (iv) public anxiety elicited by CNS infections; (v) ease with which large volumes of infectious materials can be produced; and (vi) potential for malicious introduction of foreign genes designed to increase alphavirus virulence (Sidwell et al. (2003) Antiviral Res. 57:101-11).

There are currently no specific treatments or proven cures for alphavirus infections such that reliance on host immunity responses is the standard course of care. Moreover, no FDA-approved anti-alphaviral vaccines exist. Accordingly, there is a great need in the art for effective methods for treating alphaviral infections. In addition, there is an important unmet medical need for treating such infections in cells of the CNS (e.g., neurons) without killing or reducing cellular viability since many of these cell types are terminally differentiated and will therefore not be replaced.

### SUMMARY OF THE INVENTION

The invention provides methods of inhibiting alphavirus replication and treating alphavirus infection. Accordingly, in one aspect, a method of inhibiting alphavirus replication in a medium, comprising applying to said medium an effective amount of an agent that inhibits one or more functions of a cyclophilin is provided. In some embodiments, the alphavirus is selected from the group consisting of Aura virus, Babanki, Barmah Forest virus, Bebaru virus, Buggy Creek, Cabassou virus, Chikungunya virus, Eastern equine encephalitis virus, Everglades virus, Fort Morgan virus, Getah virus, Highlands J virus, Kyzylagach virus, Mayaro virus, Middelburg virus, Mosso das Pedras virus, Mucambo virus, Ndumu virus, O'nyong-nyong virus, Pixuna virus, Rio Negro virus, Ross River virus, Sagiyama virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, and Whataroa virus. In other embodiments, the one or more functions of the cyclophilin is peptidyl prolyl isomerase enzymatic activity. In still other embodiments, the cyclophilin inhibitor inhibits the cyclophilin's peptidyl prolyl isomerase enzymatic activity and does not significantly inhibit calcineurin activity. In yet other embodiments, the cyclophilin inhibitor inhibits the cyclophilin's peptidyl prolyl isomerase enzymatic activity and does not significantly inhibit NFAT signaling. In other embodiments, the agent comprises a cyclosporin, cyclosporin derivative, salts of a cyclosporin or cyclosporin derivative, or mixtures thereof (e.g., cyclosporin A, cyclosporin A derivative, salt of cyclosporin A or cyclosporin A derivative, or mixtures thereof). In still other embodiments, the cyclophilin is selected from the group consisting of cyclophilin A, B, C, D, E, F, G, H, I, J, K, L, M, N, O, P, Q, R, S, T, U, V, W, X, Y and Z. In yet other embodiments, inhibition of alphaviral replication reduces alphaviral titers to less than 500 genome copies per milliliter of serum. In other embodiments, inhibition of alphaviral replication reduces alphaviral titers by 2-log relative to alphaviral replication in the absence of the cyclophilin inhibitor. In still other embodiments, the alphavirus replication in the medium occurs in human cells.

In another aspect, a method of treating an alphavirus infection in a subject, comprising administering to the subject an effective amount of an agent that inhibits one or more functions of a cyclophilin is provided. In some embodiments, the alphavirus is selected from the group consisting of Aura virus, Babanki, Barmah Forest virus, Bebaru virus, Buggy Creek, Cabassou virus, Chikungunya virus, Eastern equine encephalitis virus, Everglades virus, Fort Morgan virus, Getah virus, Highlands J virus, Kyzylagach virus, Mayaro virus, Middelburg virus, Mosso das Pedras virus, Mucambo virus, Ndumu virus, O'nyong-nyong virus, Pixuna virus, Rio Negro virus, Ross River virus, Sagiyama virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, and Whataroa virus. In other embodiments, the one or more functions of the cyclophilin is peptidyl prolyl isomerase enzymatic activity. In still other embodiments, the cyclophilin inhibitor inhibits the cyclophilin's peptidyl prolyl isomerase enzymatic activity and does not significantly inhibit calcineurin activity. In yet other embodiments, the cyclophilin inhibitor inhibits the cyclophilin's peptidyl prolyl isomerase enzymatic activity and does not significantly inhibit NFAT signaling. In still other embodiments, the agent comprises a cyclosporin, cyclosporin derivative, salts of a cyclosporin or cyclosporin derivative, or mixtures thereof (e.g., cyclosporin A, cyclosporin A derivative, salt of cyclosporin A or cyclosporin A derivative, or mixtures thereof). In yet other embodiments, the cyclophilin is selected from the group consisting of cyclophilin A, B, C, D, E, F, G, H, I, J, K, L, M, N, O, P, Q, R, S, T, U, V, W, X, Y and Z. In some embodiments, inhibition of alphaviral replication reduces alphaviral titers to less than 500 genome copies per milliliter of serum. In other embodiments, inhibition of alphaviral replication reduces alphaviral titers by 2-log relative to alphaviral replication in the absence of the cyclophilin inhibitor. In still other embodiments, the methods described herein further comprise administering to the subject an effective amount of at least one additional therapeutic agent (e.g., an agent that reduces alphaviral replication, reduces the time to alphaviral clearance, reduces morbidity or mortality in the clinical course of the alphaviral infection, reduces subject symptoms caused by the alphaviral infection, or reduces a side effect of the cyclophilin inhibitor). In yet other embodiments, patient symptoms caused by the alphaviral infection are selected from the group consisting of encephalitis, arthritis, rashes, fevers, headache, nausea, myalgia, arthralgia, arthropathy, chills, diarrhea, vomiting, lymphadenitis, malaise, and muscle soreness. In other embodiments, the at least one additional therapeutic agent is selected from the group consisting of a nucleoside analog, mycophenolic acid, inhibitors of inosine monophosphate dehydrogenase (IMPDH), anti-alphaviral neutralizing monoclonal antibodies, poly ICLC, triaryl pyrazolin, anti-alphaviral ribozymes, zinc-finger antiviral protein, lactoferrin, and anti-alphaviral antisense RNA inhibitors. In still other embodiments of methods described herein, the cyclophilin inhibitor and at least one additional therapeutic agent are administered together as part of a single composition or administered separately. In yet other embodiments, the administering step comprises administering the agent to the central nervous system. In some embodiments of the methods described herein, the subject is a human.

### BRIEF DESCRIPTION OF FIGURES

FIG. 1 shows sources of variation between SINV-infected and uninfected differentiated CSM14.1 cells. Source of variation analysis were generated using Partek Genomics Suite. F ratio (y-axis) reflects the relative degree to which a factor (x-axis) accounts for variance. Infection type (Type), Timepoint, and the interaction of Timepoint and Type (Timepoint*Type) are experimental factors while Scan Date and Sample are batch effects. The error term denotes the variation not accounted for in the model.

FIG. 2 shows RT-PCR results confirming differential expression of microarray-determined genes. RT-PCR of key genes identified as differently-expressed from the Cellular Effects of Sildenafil pathway are shown. ITPR3=inositol 1,4,5-tripohosphate receptor; calmodulin 2=CALM2; calmodulin 3=CALM3; phosphodiesterase 5A=PDE5A; L-type calcium channel 1D=CACNA1D.

FIGS. 3A-3C show NFAT activation state in SINV-infected, differentiated neurons. FIG. 3A shows the results of Western blot analyses of NFATc1 and p-NFATc1 in mock-infected and SINV-infected whole-cell CSM14.1. FIGS. 3B and 3C show the results of Western blot analyses of NFAT3 in subcellular fractions of SINV-infected, differentiated, treated and untreated CSM14.1. Cells were infected for 1 hour with SINV before treatment with 10 μM cyclosporin A, FK506, or zaprinast. Cells were harvested and fractionated at 3, 24, and 48 hours post-infection. M=mock-infected, mock-treated; V=SINV-infected, mock-treated; C=SINV-infected, CsA-treated; F=SINV-infected, FK506-treated; Z=SINV-infected, Zaprinast-treated. (Multiplicity of Infection (MOI)=10).

FIG. 4 shows a schematic diagram of agents that mediate NFAT activity. Cyclosporin A and FK506 form complexes with cyclophilin and FKBP1B, respectively, which inhibit calcineurin, blocking activation of NFAT. Zaprinast inhibits PDE5A, which might have a downstream effect of inducing activation of NFAT by calcineurin.

FIGS. 5A-5B show the effects of NFAT-modulating drugs on cell survival in alphaviral-infected cells. Uninfected differentiated CSM14.1 cells (dCSM14.1) were treated with DMSO, cyclosporin A, or FK506 (FIG. 5A). None of the treatments significantly reduced viability compared to untreated cells. Infected dCSM14.1 cells were treated with either cyclosporin A (10 uM) FK506 (10 uM) or zaprinast (10 uM) (FIG. 5B). Cyclosporin A significantly reduced viability of infected cells at 48 hours compared to untreated cells. **P<0.01; nonparametric t test comparing SINV-Infected, untreated with each treatment.

FIG. 6 shows the effects of NFAT-modulating drugs on alphaviral replication in alphaviral-infected cells. CSM14.1 cells were differentiated and infected for 1 hour with SINV (MOI=10) before being treated with cyclosporin A (10 uM), FK506 (10 uM), or zaprinast (10 uM). Supernatants of treated and untreated cells were collected at 0, 6, 12, 24, 36, 48 and 60 hours post infection. Viral titers were attained by plaque assay and expressed as PFU/mL. Error bars indicate standard error and mean. *P<0.05; ***P<0.001; 2-way ANOVA comparing SINV-Infected, untreated with each treatment.

FIGS. 7A-7B show the effects of NFAT-modulating drugs on alphaviral replication in alphaviral-infected cells. Experiments similar to those performed for the data shown in FIG. 6 were conducted by applying cyclosporin A (CSA) and FK506 on Sindbis virus (TE) replication in differentiated AP7 cells. ***P<0.001 for FIG. 7A) or **P<0.01 and ***P<0.001 for FIG. 7B.

### DETAILED DESCRIPTION OF THE INVENTION

The present invention is based in part on the discovery that agents that inhibit one or more functions of a cyclophilin (e.g., enzymatic activity such as cis-trans prolyl isomerase), such as cyclosporin A, also inhibit alphavirus replication in cells, such as human cells, without affecting cell viability. Accordingly, the present invention provides methods for treating an alphavirus infection in a subject suffering from an alphavirus infection, including conditions resulting from such an alphavirus infection (e.g., viral encephalitis), by administering to the individual in need thereof an effective amount of an agent that inhibits activity of a cyclophilin. In some embodiments, the methods involve administering to the individual in need thereof effective amounts of a cyclophilin inhibitor and at least one additional therapeutic agent. In other embodiments, the methods are useful for inhibiting alphavirus replication in a medium, comprising applying to said medium an effective amount of an agent that inhibits one or more functions of a cyclophilin (e.g., enzymatic activity such as cis-trans prolyl isomerase).

In order that the present invention may be more readily understood, certain terms are first defined. Additional definitions are set forth throughout the detailed description.

The term “alphavirus,” and its grammatical variants, refers to a group of Togaviridae (Group IV) family of viruses characterized by one or more of the following: (i) a positive sense, single-stranded RNA genome; (ii) RNA that is 5′ capped and 3′-polyadenylated; (iii) viral particles that are enveloped, have a 70 nm diameter, and have a 40 nm isometric nucleocapsid; (iv) viral replication in the cytoplasm of host cells; (v) lack a DNA phase during the viral replication cycle; and (vi) maintain virions that mature by budding through the plasma membrane. A non-limiting list of exemplary alphaviruses includes Aura virus (AURA), Babanki (BAB), Barmah Forest virus (BF), Bebaru virus (BEB), Buggy Creek, Cabassou virus (CAB), Chikungunya virus (CHIK), Eastern equine encephalitis virus (EEE), Everglades virus (EVE), Fort Morgan virus (FM), Getah virus (GET), Highlands J virus (HJ), Kyzylagach virus (KYZ), Mayaro virus (MAY), Middelburg virus (MID), Mosso das Pedras virus (78V3531), Mucambo virus (MUC), Ndumu virus (NDU), O'nyong-nyong virus (ONN), Pixuna virus (PIX), Rio Negro virus (AG80), Ross River virus (RR), Sagiyama virus (SAG), Salmon pancreas disease virus (SPDV), Semliki Forest virus (SF), Sindbis virus (SIN), Southern elephant seal virus, Tonate virus, Trocara virus, Una virus (UNA), Venezuelan equine encephalitis virus (VEE), Western equine encephalitis virus (WEE), and Whataroa virus (WHA). Sindbis, Semliki Forest, O'nyong'nyong, Chikungunya, Mayaro, Ross River, Barmah Forest, Eastern Equine Encephalitis, Western Equine Encephalitis, and Venezuelan Equine Encephalitis viruses are particularly relevant for medical intervention in humans. Alphaviruses are evolutionarily differentiated based on nucleotide sequence of the nonstructural proteins, of which there are four (nsP1, nsP2, nsP3 and nsP4). The genus segregates into New World (American) and Old World (Eurasian/African/Australasian) alphaviruses based on geographic distribution. It is estimated that New World and Old World viruses diverged between 2,000 and 3,000 years ago (Harley et al. (2001) Clin. Microbiol. Rev. 14:909-932). Among the alphavirus species, there are at least seven distinct serocomplexes (SF, EEE, MID, NDU, VEE, WEE and BFV) into which members of the genus are sub-divided (Khan et al. (2002) J. Gen. Virol. 83:3075-3084 and Harley et al. (2001) Clin. Microbiol. Rev. 14:909-932). Based on genomic sequence data from six of the seven serocomplexes, alphaviruses have been grouped into three large groups VEE/EEE, SFV and SIN. The VEE-EEE group is exclusively made up of New World viruses with a distribution in North America, South America and Central America.

Members of this group include EEE, VEE, EVE, MUC and PIX. The SF group is primarily Old World, but contains one member (MAY) that is found in South America. Other members of the SF group include SF, MID, CHIK, ONN, RR, BF, GET, SAG, BEB and UNA. The SIN group is also primarily Old World, with the exception of AURA, which is a New World virus related to SIN and can be found in Brazil and Argentina. Other members of this group include SIN, WHA, BAB and KYZ. WEE, HJ and FM are considered recombinant viruses and are thus not included in any of the three groups. Table 1 below provides additional detail of alphaviruses, especially with regard to human diseases, reservoir hosts, and geographic distribution.

TABLE 1

| | | Principal | | | |
|---|---|---|---|---|---|
| | Antigenic | Vertebrate | Geographic | | |
| Virus (abbreviation) | Complex | Reservoir Host | Distribution | Human Disease | Animal Disease |
| Aura (AURA) | WEE | ? | S. Amer. | | |
| Barmah Forest (BF) | BF | birds | Australia | Fever, Arthritis, Rash | |
| Bebaru (BEB) | SF | ? | Asia | | |
| Cabassou (CAB) | VEE | ? | French Guiana | | |
| Chikungunya (CHIK) | SF | primates | Africa, SE Asia, | Fever, Arthritis, Rash | |
| | | | Philippines, Indonesia | | |
| Eastern equine | EEE | birds | N. Amer., S. Amer., | Fever, Encephalitis | horse pheasant, emu, |
| encephalitis (EEE) | | | Caribbean | | pigeon, turkey |
| Everglades (EVE) | VEE | mammals | Florida | Fever, Encephalitis | |
| Fort Morgan (FM) | WEE | birds | Colorado | | |
| Getah (GET) | SF | mammals | Asia | Fever | horse |
| Highlands J (HJ) | WEE | birds | N. Amer. | | horse, turkey, emu, |
| | | | | | pheasant, duck, crane |
| Mayaro (MAY) | SF | mammals | S. Amer. | Fever, Arthritis, Rash | |
| Middelburg (MID) | MID | ? | Africa | | |
| Mosso das | VEE | ? | S. Amer. | | |
| Pedras/78V3531 | | | | | |
| Mucambo (MUC) | VEE | ? | S. America, Caribbean | | |
| Ndumu (NDU) | NDU | ? | Africa | | |
| O'nyong-nyong | SF | ? | East Africa | Fever; Arthritis, Rash | |
| (ONN) | | | | | |
| Pixuna (PIX) | VEE | mammals | Brazil | | |
| Rio Negro (AG80) | VEE | mammals | Argentina | | |
| Ross River (RR) | SF | mammals | Australia, S. Pacific | Fever, Arthritis, Rash | |
| Salmon pancreas | ? | fish | North Atlantic | | Trout, salmon |
| disease | | | | | |
| Semliki Forest (SF) | SF | ? | Africa | Fever, Encephalitis | horse |
| Sindbis (SIN) | WEE | birds | Australia, Africa, N. | Fever, Arthritis, Rash | |
| | | | Europe, Middle East | | |
| Southern Elephant | ? | seals | Antarctica | | |
| Seal (SES) | | | | | |
| Tonate | VEE | ? | S. Amer. | Fever, Encephalitis | |
| Trocara | | ? | S. Amer. | | |
| Una (UNA) | SF | ? | S. Amer., Trinidad | | horse |
| Venezuelan equine | VEE | mammals | S. Amer., N. Amer. | Fever, Encephalitis | horse |
| encephalitis (VEE) | | | | | |
| Western equine | WEE | birds, mammals | N. Amer., S. Amer. | Fever, Encephalitis | horse, emu |
| encephalitis (WEE) | | | | | |
| Whataroa (WHA) | WEE | birds | New Zealand, | | |
| | | | Australia | | |

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

The term “binding” or “interacting” refers to an association, which may be a stable association, between two molecules, e.g., between a cyclophilin and an agent that inhibits the cyclophilin's cis-trans prolyl isomerase activity (e.g., prolyl isomerization and/or protein folding mediated by cyclophilin) due to, for example, electrostatic, hydrophobic, ionic and/or hydrogen-bond interactions under physiological conditions. Exemplary interactions include protein-protein, protein-nucleic acid, protein-small molecule, and small molecule-nucleic acid interactions. In some embodiments, a cyclosporin is considered to bind to cyclophilin if it binds to human recombinant cyclophilin at least 20%, 25%, 30%, 35%, 40%, 45%, 50%, 55%, 60%, 65%, 70%, 75%, 80%, 85%, 90%, 95% as well as does cyclosporin A in the competitive ELISA test described by Quesniaux (1987) Eur. J. Immunol. 17:1359-1365. In this assay, the cyclosporin to be tested is added during the incubation of cyclophilin with coated BSA-cyclosporin and the concentration required to give a 50% inhibition of the control reaction without competitor is calculated (IC₅₀). The results are expressed as the Binding Ratio (BR), which is the log to the base 10 of the ratio of the IC₅₀ of the test compound and the IC₅₀ in a simultaneous test of the cyclosporin itself. Thus, a BR of 1.0 indicates that the test compound binds human cyclophilin one factor of ten less well than does the cyclosporin, and a negative value indicates binding stronger than that of the cyclosporin. The cyclosporine active against an alphavirus can have a BR lower than 0.7, 0.6, 0.5, 0.4, 0.3, 0.2, 0.1 or lower than zero.

The term “biological sample” is intended to include tissues, cells and biological fluids isolated from a subject, as well as tissues, cells and fluids present within a subject.

The term “controlled drug delivery device” is meant to encompass any device wherein the release (e.g., rate or timing of release) of a drug or other desired substance contained therein is controlled by or determined by the device itself and not substantially influenced by the environment of use, or releasing at a rate that is reproducible within the environment of use. By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug, received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

The term “cyclophilin” refers to a family of proteins that bind to cyclosporine and have peptidyl prolyl isomerase activity, which catalyzes the isomerization of peptide bonds from trans from to cis form at proline residues and facilitates protein folding. Members of this family are well known in the art and include, for example, cyclophilin A (e.g., Human Gene ID 5478; RefSeq mRNA NM_{—}021130.3, and RefSeq protein NP_{—}066953.1; Mouse Gene ID 268373; RefSeq mRNA XM_{—}001002180.1, and RefSeq protein NP_{—}032933.1), cyclophilin B (e.g., Human Gene ID 5479; RefSeq mRNA NM_{—}000942.4, and RefSeq protein NP_{—}000933.1; Mouse Gene ID 19035; RefSeq mRNA NM_{—}011149.2, and RefSeq protein NP_{—}035279.2), cyclophilin C (e.g., Human Gene ID 5480; RefSeq mRNA NM_{—}000943.4, and RefSeq protein NP_{—}0000934.1; Mouse Gene ID 19038; RefSeq mRNA NM_{—}008908.4, and RefSeq protein NP_{—}032934.1), and cyclophilin D (e.g., Human Gene ID 5481; RefSeq mRNA NM_{—}005038.2, and RefSeq protein NP_{—}005029.1; Mouse Gene ID 67738; RefSeq mRNA NM_{—}026352.3, and RefSeq protein NP_{—}080628.1).

The term “cyclophilin inhibitor” refers to any agent that inhibits one or more functions of a cyclophilin, such as enzymatic activity of a cyclophilin. The term includes, but is not limited to, agents that inhibit cyclophilin cis-trans prolyl isomerase activity; agents that inhibit protein folding rates or substrates mediated by the cyclophilin; and agents that bind to cyclophilin but do not cause immunosuppression and/or mediate and/or activate calcineurin or NFAT signaling.

The term “cyclosporin” refers to a family of cyclic poly-amino acid molecules. Cyclosporins generally are poly-N-methylated undecapeptides commonly possessing immunosuppressive or anti-inflammatory properties. The family of cyclosporine includes cyclosporin A, as well as cyclosporin B, C, D, E, F, G, H, I, J, K, L, M, N, O, P, Q, R, S, T, U, V, W, X, Y and Z (see, for example, Traber et al. (1977) Helv. Chim. Acta 60:1247-1255; Traber et al. (1982) Helv. Chim. Acta 65:1655-1667; Kobel et al. (1982) Eur. J. Appl. Microbiol. Biotech. 14:237-240; and von Wartburg et al. (1986) Prog. Allergy 38:28-45). Some known metabolites of cyclosporin A include the following: (according to Hawk's Cay nomenclature) AM1, AM9, AM1c, AM4N, AM19, AM1c9, AM1c4N9, AM1A, AM1A4N, AM1Ac, AM1AL, AM11d, AM69, AM4N9, AM14N, AM14N9, AM4N69, AM99N, Dihydro-CsA, Dihydro-CsC, Dihydro-CsD, Dihydro-CsG, M17, AM1c-GLC, sulphate conjugate of cyclosporin, BH11a, BH15a, B, G, E, (and with come overlap with the Hawk's above, according to Maurer's nomenclature) M1, M2, M3, M4, M5, M6, M7, M8, M9, M10, M11, M12, M13, M14, M15, M16, M17, M18, M19, M20, M21, M22, M23, M24, M25, M26, MUNDF1 and MeBMT. Some metabolites of cyclosporin G include GM1, GM9, GM4N, GM1c, GM1c9, and GM19. Modified cyclosporins include modified C-9 amino acid analogs, modified 8-amino acid analogs, modified 6-position analogs containing MeAla or MeAbu residue, and SDZ 209-313, SDZ-205-549, SDZ-033-243, and SDZ-PSC-833. Such compounds, including methods for making, formulating, administering for immunosuppression purposes, and the like, are well known in the art (see, for example, U.S. Pat. Nos. 4,117,118; 6,254,860; 6,350,442; and numerous patents since). Unless otherwise stated, the entire family of cyclosporins, including cyclosporin A, all derivatives, variants, amino acid variants, metabolites, including variations of mono-, di- and trihydroxylates, N-demethylates, aldehydes, carboxylates, conjugates, sulphates, glucuronides, intramolecular cyclizations and those without a cyclic structure as well as shorter peptides and amino acids and their derivatives and salts with or without immunosuppressive properties and whether able to cross the blood brain barrier or not will hereinafter be referred to as cyclosporin or cyclosporins. The term “cyclosporin A” refers to a product of the fungus Tolypocladium Inflatum. It is a cyclic poly-amino acid molecule, consisting of 11 amino acids. One of the amino acids is unique for cyclosporin A, a β-hydroxyamino acid called butenyl-methyl-threonin (MeBmt). The molecular weight is 1202.6 and the chemical composition is C₆₂H₁₁₁N₁₁O₁₂. Also included in this definition is all cyclosporin A derivatives, variants, amino acid variants, metabolites, including variations of mono-, di- and trihydroxylates, N-demethylates, aldehydes, carboxylates, conjugates, sulphates, glucuronides, intramolecular cyclizations and those without a cyclic structure as well as shorter peptides and amino acids and their derivatives and salts with or without immunosuppressive properties and whether able to cross the blood brain barrier or not will. The same scope of coverage applies for other individual cyclosporins, such as cyclosporin B, cyclosporin C, etc.

The term “diagnosed,” as used herein, refers to the recognition of a disease by its signs and symptoms (e.g., resistance to conventional therapies), or genetic analysis, pathological analysis, histological analysis, diagnostic assay (e.g., for alphavirus infection) and the like.

The term “dosing event” refers to administration of an antiviral agent to a subject in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system. “Continuous delivery” (e.g., in the context of “continuous delivery of a substance to a tissue”) refers to movement of drug to a delivery site, e.g., into a tissue in a fashion that provides for delivery of a desired amount of substance into the tissue over a selected period of time, where about the same quantity of drug is received by the patient each minute during the selected period of time. “Controlled release” (e.g., in the context of “controlled drug release”) refers to release of an agent, such as a cyclophilin inhibitor, at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

The term “encephalitis” refers to an acute inflammation of the brain. Viral encephalitis can occur either as a direct effect of a viral infection or as one of the sequelae of a latent infection.

The term “flavivirus” includes any member of the family Flaviviridae, including, but not limited to, Dengue virus, including Dengue virus 1, Dengue virus 2, Dengue virus 3, Dengue virus 4 (see, e.g., GenBank Accession Nos. M23027, M19197, A34774, and M14931); Yellow Fever Virus; West Nile Virus; Japanese Encephalitis Virus; St. Louis Encephalitis Virus; Bovine Viral Diarrhea Virus (BVDV); Tick-Borne Encephalitis Virus; and Hepatitis C Virus; and any serotype, strain, genotype, subtype, quasispecies, or isolate of any of the foregoing. Although similar to alphaviruses, flaviviruses differ from alphaviruses by at least one or more of the following: (i) the genomic RNA is not polyadenylated until it enters the host cell; (ii) the single capsid protein is smaller (i.e., approximately 14 kD rather than the approximately 30 kD capsid protein of alphaviruses); and (iii) the mature virion forms in the cytoplasm in association with the endoplasmic reticulum instead of budding from the cellular plasma membrane. The class of flaviviruses are distinct and severable from the class of alphaviruses.

The terms “individual,” “host,” “subject,” and “patient” are used interchangeably herein, and refer to a mammal, including, but not limited to, primates, including simians and humans.

The term “isolated polypeptide” refers to a polypeptide, in certain embodiments prepared from recombinant DNA or RNA, or of synthetic origin, or some combination thereof, which (1) is not associated with proteins that it is normally found within nature, (2) is isolated from the cell in which it normally occurs, (3) is isolated free of other proteins from the same cellular source, (4) is expressed by a cell from a different species, or (5) does not occur in nature.

The terms “label” or “labeled” refer to incorporation or attachment, optionally covalently or non-covalently, of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

The term “patterned” or “temporal” as used in the context of drug delivery refers to delivery of drug in a pattern, generally a substantially regular pattern, over a pre-selected period of time (e.g., other than a period associated with, for example a bolus injection). “Patterned” or “temporal” drug delivery encompasses delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “substantially steady state” as used in the context of a biological parameter that may vary as a function of time, refers to a biological parameter that exhibits a substantially constant value over a time course, e.g., such that the area under the curve defined by the value of the biological parameter as a function of time for any 8 hour period during the time course (AUC8 hr) is no more than about 20% above or about 20% below, and preferably no more than about 15% above or about 15% below, and more preferably no more than about 10% above or about 10% below, the average area under the curve of the biological parameter over an 8 hour period during the time course (AUC8 hr average). The AUC8 hr average is defined as the quotient (q) of the area under the curve of the biological parameter over the entirety of the time course (AUCₜₒₜₐₗ) divided by the number of 8 hour intervals in the time course (t_{total1/3 days)}, i.e., q=(AUCₜₒₜₐₗ)/(t_{total1/3 days}). For example, in the context of a serum concentration of a drug, the serum concentration of the drug is maintained at a substantially steady state during a time course when the area under the curve of serum concentration of the drug over time for any 8 hour period during the time course (AUC8 hr) is no more than about 20% above or about 20% below the average area under the curve of serum concentration of the drug over an 8 hour period in the time course (AUC8 hr average), i.e., the AUC8 hr is no more than 20% above or 20% below the AUC8 hr average for the serum concentration of the drug over the time course.

The term “sustained viral response” (SVR; also referred to as a “sustained response” or a “durable response”), as used herein, refers to the response of an individual to a treatment regimen for alphaviral infection, in terms of serum alphaviral titer. Generally, a “sustained viral response” refers to no detectable alphaviral RNA (e.g., less than about 1,000, 900, 800, 700, 600, 500, 400, 300, 200, 100 or less genome copies per milliliter serum; or less than about 10, 9, 8, 7, 6, 5, 4, 3, 2, 1 or less log₁₀ plaque forming unites (pfu) per milliliter serum) found in the patient's serum for a period of at least about one month, at least about two months, at least about three months, at least about four months, at least about five months, or at least about six months following cessation of treatment.

The term “therapeutically effective amount” refers to an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art. In certain embodiments, the therapeutically effective amount of a cyclophilin inhibitor in combination with a second therapeutic agent is an amount that is synergistic. As used herein, a “synergistic combination” or a “synergistic amount” of an inhibitor of cyclophilin inhibitor and a second therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the cyclophilin inhibitor when administered at that same dosage as a monotherapy and (ii) the therapeutic or prophylactic benefit of the second therapeutic agent when administered at the same dosage as a monotherapy.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease, such as in encephalitis resulting in the context of alphavirus infection; (b) inhibiting the disease (i.e., arresting its development); and (c) relieving the disease (i.e., causing regression of the disease).

The term “treatment failure subjects” (or “treatment failures”) as used herein generally refers to alphavirus-infected subjects who previously failed to respond to anti-alphaviral therapy alphaviral (referred to as “non-responders”) or who initially responded to previous therapy, but in whom the therapeutic response was not maintained (referred to as “relapsers”). The previous therapy generally can include treatment with antiviral agents and/or agents to ameliorate the systems of the alphaviral infection (e.g., anti-inflammatory agents to reduce arthritis).

Before the present invention is further described, it is to be understood that this invention is not limited to particular embodiments described, as such may, of course, vary. It is also to be understood that the terminology used herein is for the purpose of describing particular embodiments only, and is not intended to be limiting. The methods and compositions described herein are generally useful in treatment of any alphavirus. Treatment of alphaviral infection is of particular interest in some embodiments. Reference to particular alphaviruses herein is for illustration only and is not meant to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a cyclophilin inhibitor” includes a plurality of such inhibitors and reference to “the active agent” includes reference to one or more active agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods of Treating Alphaviral Infections

The present invention provides monotherapy and combination therapy methods of treating an alphavirus infection. In some embodiments, the present invention provides methods of reducing the incidence of complications associated with alphaviral infection; and methods of reducing viral load, or reducing the time to viral clearance, or reducing morbidity or mortality in the clinical outcomes, in subjects suffering from alphaviral infection. In some embodiments, such methods are amenable to treatment in a subject (e.g., a human patient). In other embodiments, such methods are applicable to alphaviruses present in a medium. The methods generally involve administering to a culture containing an alphavirus or a subject an effective amount of an agent that inhibits one or more functional activities (e.g., enzymatic activities) of a cyclophilin via a monotherapy or a combination therapy. Effective amounts of an agent that inhibits enzymatic activity of a cyclophilin, as well as dosing regimens, are discussed below.

In general, an effective amount of an agent that inhibits one or more functional activities (e.g., enzymatic activities) of a cyclophilin is an amount that is effective to reduce viral replication; or reduce the time to viral clearance; or an amount that is effective to reduce morbidity or mortality in the clinical course of the disease; or an amount that is effective to improve some other indicator of disease response (e.g., an amount that is effective to reduce viral load; achieve a sustained viral response; etc.).

In some embodiments, a subject monotherapy treatment method is effective to decrease viral load in the individual, and to achieve a sustained viral response. Of particular interest in many embodiments is treatment of humans.

In one embodiment, the method involves administering an effective amount of an agent that inhibits one or more functions (e.g., enzymatic activity) of a cyclophilin. Whether a subject monotherapy method is effective in treating an alphaviral infection can be determined by measuring viral load, or by measuring a parameter associated with alphaviral infection, including, but not limited to, encephalitis, arthritis, rashes, fevers, headache, nausea, myalgia, arthralgia (joint pain), arthropathy (diseases of the joint), chills, diarrhea, vomiting, lymphadenitis, malaise, and muscle soreness, according to well known methods in the art.

In some embodiments, an effective amount of an agent that inhibits one or more functional activities (e.g., enzymatic activities) of a cyclophilin is an amount that is effective to reduce viral titers to undetectable levels, e.g., to about 1000 to about 5000, to about 500 to about 1000, or to about 100 to about 500 genome copies/mL serum. In some embodiments, an effective amount of an agent that inhibits enzymatic activity of a cyclophilin is an amount that is effective to reduce viral load to lower than 100 genome copies/mL serum.

In some embodiments, an effective amount of an agent that inhibits one or more functional activities (e.g., enzymatic activities) of a cyclophilin is an amount that is effective to achieve a 1.5-log, a 2-log, a 2.5-log, a 3-log, a 3.5-log, a 4-log, a 4.5-log, or a 5-log reduction in viral titer in the serum of the individual. Whether a subject method is effective in treating an alphaviral infection can be determined by a reduction in number or length of hospital stays, a reduction in time to viral clearance, a reduction of morbidity or mortality in clinical outcomes, a reduction in viral burden, or other indicator of disease response in the patient.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of alphaviral RNA have been developed. Many such assays are available commercially, including a quantitative reverse transcription PCR(RT-PCR) (Amplicor alphaviral Monitor™, Roche Molecular Systems, New Jersey); and a branched DNA (deoxyribonucleic acid) signal amplification assay (Quantiplex™ alphaviral RNA Assay (bDNA), Chiron Corp., Emeryville, Calif.). See, e.g., Gretch et al. (1995) Ann. Intern. Med. 123:321-329.

In some embodiments, the methods of the invention achieve a sustained viral response, e.g., the viral load is reduced to undetectable levels for a period of at least about one month, at least about two months, at least about three months, at least about four months, at least about five months, or at least about six months following cessation of treatment.

Whether a method described herein is effective in treating an alphaviral infection can be determined by measuring a parameter associated with alphaviral infection, such as viral load, arthritis, encephalitis, rashes, fevers, headaches, nausea, myalgia, arthralgia (joint pain), arthropathy (diseases of the joint), chills, diarrhea, vomiting, lymphadenitis, malaise, and muscle soreness.

Generally, the methods described herein are suitable for treating subjects having, or susceptible to having an alphavirus infection. The subject methods are also suitable for treating subjects who have been previously treated for an alphavirus infection with an agent other than a cyclophilin inhibitor and are refractory to treatment with the agent, and who have either failed the previous treatment; or who cannot tolerate treatment with the non-cyclophilin agent; or who responded to the previous treatment and relapsed. In many embodiments, the individual is a human.

Subjects who have been clinically diagnosed as infected with an alphavirus are suitable for treatment with a method of the instant invention. Of particular interest in some embodiments are subjects who have been clinically diagnosed as infected with an alphavirus (e.g., clinically diagnosed as having alphaviral RNA and/or anti-alphaviral antibody in their blood or serum.

Subjects who are clinically diagnosed as infected with alphaviral include naive subjects (e.g., subjects who have not previously treated for alphaviral infection) and subjects who have failed prior treatment for alphaviral (“treatment failure” subjects) can be treated using the methods described herein. Treatment failure subjects include non-responders (i.e., subjects in whom the alphaviral titer was not significantly or sufficiently reduced by a previous treatment for alphaviral infection and relapsers. Also of interest are alphaviral-positive subjects (as described above) who exhibit symptoms consistent with alphaviral infection but who have not yet been clinically diagnosed or who are viremic despite prior antiviral treatments or who have a contraindication to standard antiviral treatments.

### Agents Suitable for Use in Monotherapy

Agents that are suitable for use in a subject treatment method are agents that inhibits the activity of one or more functions of a cyclophilin (e.g., cyclophilin A). In some embodiments, the agent is one that inhibits enzymatic activity of the cyclophilin. An agent that is suitable for use in a subject monotherapy is an agent the can inhibit the one or more functions of a cyclophilin by at least about 10%, at least about 15%, at least about 20%, at least about 25%, at least about 30%, at least about 35%, at least about 40%, at least about 50%, at least about 60%, at least about 70%, at least about 80%, or at least about 90%, or more, compared to the activity of the cyclophilin in the absence of the agent. A suitable agent is an agent that preferentially inhibits the one or more functions of a cyclophilin, e.g., the agent inhibits enzymatic activity of a cyclophilin preferentially, compared to the inhibition, if any, by the agent of a cyclophilin. In other words, a suitable agent inhibits enzymatic activity of a cyclophilin and if the agent inhibits a cyclophilin at all, the agents inhibits less than about 20%, less than about 15%, less than about 10%, less than about 5%, less than about 2%, or less than about 1%, of the activity of a cyclophilin.

In some embodiments, agents that are specifically excluded from use in a subject monotherapy include agents that cause immunosuppression and/or inhibit calcineurin activity and/or NFAT signaling, such as FK-506.

In other embodiments, a suitable agent is a selective inhibitor of a cyclophilin. The term “selective inhibitor of a cyclophilin” is used herein to mean an agent which selectively inhibits cyclophilin enzymatic activity in preference to or in exclusion to causing/affecting immunsuppression and/or calcineurin activity and/or NFAT signaling. In some embodiments, the selective inhibitor of a cyclophilin has a ratio of the IC₅₀ concentration (concentration inhibiting 50% of enzymatic activity) for the cyclophilin to the IC₅₀ concentration for affecting calcineurin activity and/or NFAT signaling by the cyclophilin is less than 1, 0.9, 0.8, 0.7, 0.6, 0.5 or less.

Of particular interest in some embodiments of a subject monotherapy is use of an agent that inhibits one or more functions (e.g., enzymatic activity) of a cyclophilin with an IC₅₀ of less than about 50 μM, e.g., a suitable agent inhibits enzymatic activity of a cyclophilin with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less.

In many embodiments, an agent that inhibits one or more functions (e.g., enzymatic activity) of a cyclophilin also inhibits alphaviral replication. For example, an agent that inhibits enzymatic activity of a cyclophilin inhibits viral replication by at least about 10%, at least about 15%, at least about 20%, at least about 25%, at least about 30%, at least about 40%, at least about 50%, at least about 60%, at least about 70%, at least about 80%, or at least about 90%, or more, compared to alphaviral replication in the absence of the compound. Whether a compound inhibits viral replication can be determined using methods known in the art, including an in vitro viral replication assay.

In some embodiments, the agent is a cyclosporin. The term “cyclosporin” has been defined above. In other embodiments, cyclosporins having decreased immunosuppressive activity while maintaining the ability to inhibit one or more functions (e.g., enzymatic activity) of a cyclosporin are useful. Such cyclosporins are well known in the art (see, for example, WO 98/28328, WO 98/28329, WO 98/28330, WO 00/01715, and EP 0484281).

Current medical uses of cyclosporin A relate to the ability of this compound to suppress the cell-mediated immune response by preventing production and release of several autocrine T-cell growth factors, including interleukin 2 (IL-2), from activated T cells (see Borel (1989) Transplant. Proceed. 21, 810-815; Kronke et al. (1984) Proc. Natl. Acad. Sci. USA 81, 5214-5218; and Faulds et al. (1993) Drugs 45, 953-1040). Upon entry into cells, Cyclosporin A binds to cyclophilins with high affinity (see Handschumacher et al. (1984) Science 226, 544-547). As among different biological function, they have peptidyl-prolyl cis-trans isomerase (PPlase) activity that can be measured in vitro (see Fischer et al. (1989) Nature 337, 476-478; Takahashi et al. (1989) Nature 337, 473-475). Critical for the immunosuppressive effect of cyclosporin A is an interaction between cyclophilin-Cyclosporin A complex and calcium- and calmodulin-dependent serine/threonine phosphatase 2B (calcineurin) (see Hauske (1993) DN&P 6, 705-711, Friedman et al. (1991) Cell 66, 799-806; Liu et al. (1991) Cell 66, 807-815). Formation of this ternary complex results in an inhibition of the phosphatase activity of calcineurin. (see Jain et al. (1993) Nature 365, 352-355; Rao et al. (1997) Annu Rev. Immunol. 15, 707-747; Crabtree (1999) Cell 96, 611-614). Calcineurin promotes the selective dephosphorylation of NF-AT that then translocates to the nucleus where it associates with activator protein 1 and transactivates target genes, including the IL-2 gene. Cyclophilins having reduce immunosuppressive activity have been engineered by reducing the agent's ability to interact with calcineurin, as shown by transcriptional and immunological assays as well as a significantly increased affinity for cyclophilins as indicated by assays of inhibition of peptidyl-prolyl cis-trans isomerase activity.

Peptidyl-prolyl cis-trans isomerase (PPlase) activity of cyclophilins can be determined using a procedure adapted from Kofron et al. (see Biochemistry 30, 6127-6134 (1991); J. Am. Chem. Soc. 114, 2670-2675 (1992)). Similarly, an NFAT-dependent reporter assay can be used to estimate immunosuppressive activities of cyclosporins as described in Baumann et al. (1992) Transplant. Proc. 24, 43-48.

In some embodiments, cyclosporins can be used to treat alphaviral infections of the central nervous system (CNS). However, the treatment of CNS infections presents an additional hurdle to overcome, as the blood-brain-barrier (BBB) represents a formidable obstacle for drug penetration (Pardridge (2005) NeuroRx 2:1-2). The BBB is a highly effective physiologic barrier whose primary function is to closely regulate access of blood stream components to the CNS. Although infectious and inflammatory CNS diseases often disrupt BBB function and increase permeability, drug penetration remains an important aspect to consider in the development of antiviral agents against neurotropic alphaviruses. Multiple physical and chemical factors influence CNS penetration of drugs, including lipophilicity, ionization properties, molecular flexibility, polar surface area (PSA), and size (Pajouhesh et al. (2005) NeuroRx 2:541-553). Also, cyclosporins are highly lipophilic and virtually insoluble in water. They require an emulsifier to remain in aqueous phase, such as cremophore or labrafil, which are anaphylactic and neurotoxic. In some embodiments requiring administration to the CNS or to neural cells, cyclosporin formulations can be used that are neither anaphylactic nor neurotoxic. Such cyclosporin formulations are known in the art (see, for example, U.S. Pat. No. 7,446,093) and can also appreciably penetrate the blood-brain barrier.

Whether treatment with an agent that inhibits one or more cyclophilin functions (e.g., enzymatic activity) is effective in treating symptoms of alphaviral infection (e.g., encephalitis, arthritis, rashes, fevers, headache, nausea, myalgia, arthralgia (joint pain), arthropathy (diseases of the joint), chills, diarrhea, vomiting, lymphadenitis, malaise, and muscle soreness) can be determined by any of a number of techniques well known in the art. In some embodiments, an effective amount of an agent that inhibits one of more cyclophilin functions (e.g., enzymatic activity) is an amount that is effective to reduce a symptom of alphaviral infection (e.g., encephalitis, arthritis, rashes, fevers, headache, nausea, myalgia, arthralgia (joint pain), arthropathy (diseases of the joint), chills, diarrhea, vomiting, lymphadenitis, malaise, and muscle soreness) by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, or more, compared to that in an untreated subject, or to a placebo-treated subject. Those skilled in the art can readily measure such indices of alphaviral symptoms, using standard assay methods, many of which are commercially available, and are used routinely in clinical settings.

Indirect markers of alphaviral infection symptoms can also be measured as an indication of the efficacy of a subject treatment method. For example, markers of inflammation, such as TNF-alpha, IL-1 beta, and IL-20, can be analyzed as an indirect marker of arthritis. Those skilled in the art can readily measure such markers using standard assay methods, many of which are commercially available, and are used routinely in clinical settings. Methods of measuring such markers include nucleic acid-based detection methods and immunological-based methods, e.g., enzyme-linked immunosorbent assays (ELISA), radioimmunoassays, and the like, using an antibody specific for a given serum marker.

The effectiveness of any particular cyclophilin inhibitor or second therapeutic agent to treat alphaviral infection can be monitored by comparing two or more samples obtained from a subject undergoing treatment. In general, it is preferable to obtain a first sample from the subject prior to beginning therapy and one or more samples during treatment. In such a use, a baseline of antiviral effects of cells from subjects infected with an alphavirus prior to therapy is determined and then changes in the baseline state of expression of cells from subjects infected with the alphavirus is monitored during the course of therapy. Alternatively, two or more successive samples obtained during treatment can be used without the need of a pre-treatment baseline sample. In such a use, the first sample obtained from the subject is used as a baseline for determining whether the expression of cells from subjects with obesity or obesity-related disorders is increasing or decreasing.

### Combination Therapies

The present invention further provides combination therapies. Although no FDA-approved therapeutics or vaccines have been approved specifically to target alphaviral infection, the present invention provides methods for combining cyclophilin inhibitors with at least a second therapeutic agent. In some embodiments, the at least a second therapeutic agent is a therapeutic that inhibits viral replication.

In other embodiments, the at least a second therapeutic is a therapeutic that ameliorates one or more symptoms of alphaviral infection. As with monotherapy methods described above, combination therapies generally involve administering to a subject in need thereof an effective amount of an agent that inhibits one or more functions of a cyclophilin (e.g., enzymatic activity), in combination with at least a second therapeutic agent. In some embodiments, the second therapeutic agent is effective to reduce viral replication, or reduce the time to viral clearance, or an amount that is effective to reduce morbidity or mortality in the clinical course of the disease, or an amount that is effective to improve some other indicator of disease response (e.g., an amount that is effective to reduce viral load; achieve a sustained viral response; etc.).

A subject combination therapy involves administering effective amounts of a cyclophilin inhibitor and at least a second therapeutic agent. In some embodiments, effective amounts of a cyclophilin inhibitor and at least a second therapeutic agent are amounts that, in combination therapy, are effective to reduce viral titers to undetectable levels, e.g., to about 1000 to about 5000, to about 500 to about 1000, or to about 100 to about 500 genome copies/mL serum. In some embodiments, effective amounts of a cyclophilin inhibitor and at least a second therapeutic agent are amounts that, in combination therapy, are effective to reduce viral load to lower than 100 genome copies/mL serum.

In some embodiments, effective amounts of a cyclophilin inhibitor and at least a second therapeutic agent are amounts that, in combination therapy, are effective to achieve a 1.5-log, a 2-log, a 2.5-log, a 3-log, a 3.5-log, a 4-log, a 4.5-log, or a 5-log reduction in viral titer in the serum of the individual.

In other embodiments, effective amounts of a cyclophilin inhibitor and at least a second therapeutic agent are amounts that, in combination therapy, are synergistic amounts. In some of these embodiments, the amount of the at least one second therapeutic agent that is required to be administered to the individual to achieve a desired therapeutic effect (e.g., reduction in serum viral load) is reduced, compared to the dose that is normally required to be administered to achieve the same effect (e.g., the same reduction in serum viral load), when the at least one additional second therapeutic agent is administered in monotherapy or in the absence of co-administration with a cyclophilin inhibitor. As one non-limiting example, a reduction in alphaviral serum viral load can be achieved using a combination of a cyclophilin inhibitor and IFNα, where the amount of IFNα in the combination therapy that is required to achieve the reduction in the serum alphaviral viral load is lower than the amount of IFNα that would be required to achieve the same reduction in alphaviral serum viral load were the IFNα administered in monotherapy. In some of these cyclophilin inhibitor/second therapeutic agent combination therapy embodiments, the amount of the cyclophilin inhibitor that is required to be administered to the individual to achieve a desired therapeutic effect (e.g., reduction in serum viral load) is reduced, compared to the dose that is normally required to be administered to achieve the same effect (e.g., the same reduction in serum viral load), when the cyclophilin inhibitor is administered in monotherapy. In some of these cyclophilin inhibitor/second therapeutic agent combination therapy embodiments, the amount of the cyclophilin inhibitor and the amount of the second therapeutic agent that are required to be administered to the individual to achieve a desired therapeutic effect (e.g., reduction in serum viral load) are reduced, compared to the doses that are required to achieve the same effect (e.g., the same reduction in serum viral load) in monotherapy, e.g., when the cyclophilin inhibitor is administered in monotherapy, and when the second therapeutic agent is administered in monotherapy.

Suitable second therapeutic agents for treating an alphavirus infection include, but are not limited to, a nucleoside analog (e.g., ribavirin, levovirin, viramidine), mycophenolic acid, inhibitors of inosine monophosphate dehydrogenase (IMPDH); anti-alphaviral, neutralizing monoclonal antibodies (see, for example, U.S. Pat. No. 6,812,329); interferon alpha (see, for example, US Pat. Publ. 2006/0024270); poly ICLC (i.e., a synthetic complex of carboxymethylcellulose, polyinosinic-polycytidylic acid, and poly-L-lysine double-stranded RNA; Wong et al. (2005) Vaccine 23:2266-2268); triaryl pyrazolin (see, for example, Puig-Basagoiti et al. (2006) Antimicrob. Agents Chemother. 50:1320-1329); ribozymes that are complementary to viral nucleotide sequences (see, for example, Seyhan et al. (2002) J. Biol. Chem. 277:25957-25962); zinc-finger antiviral protein (see, for example, Bick et al. (2003) J. Virol. 77:11555-11562); human lactoferrin (Waarts et al. (2005) Virol. 333:284-292); antisense RNA inhibitors; and the like.

For example, nucleoside analogs for use as antiviral agents are well known in the art. Nucleoside analogs that are suitable for use in a subject combination therapy include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 143-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuraosylguanine, 9-β-L-ribofuranosyl-6-thioguanine, 2-amino-α-L-ribofuran[1′,2′:4,5]oxazoline, O.sup.2, O.sup.2-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O.sup.2, O.sup.2-anhydro-β-L-arabinofuranosyluracil, 2′-deoxy-β-L-uridine, 3′5′-Di-O-benzoyl-2′ deoxy-4-thio β-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula I of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like. The nucleoside analogs may be administered orally in capsule or tablet form, or in the same or different administration form and in the same or different route as the cyclophilin inhibitor. Of course, other types of administration of both medicaments, as they become available are contemplated, such as by nasal spray, transdermally, by suppository, by sustained release dosage form, etc. Any form of administration will work so long as the proper dosages are delivered without destroying the active ingredient.

In some embodiments, mycophenolic acid (Malinoski et al. (1981) Virol. 110:281-289), carbodine, triaryl pyrazoline (Puig-Basagoiti et al. (2006) Antimicrob. Agents Chemother 50:1320-1329) or seco-pregnane steroids from the Chinese herbs Strobilanthes cusia and Cynanchum paniculatum (Li et al., (2007) Proc. Natl. Acad. Sci. USA 104:8083-8038) can be used to inhibit alphavirus replication.

IMPDH inhibitors that are suitable for use in a subject combination therapy include, but are not limited to, VX-497 ((S)—N-3-[3-(3-methoxy-4-oxazol-5-yl-phenyl)-ureido]-benzyl-carbamic acid tetrahydrofuran-3-yl-ester); Vertex Pharmaceuticals; see, e.g., Markland et al. (2000) Antimicrob. Agents Chemother. 44:859-866); and the like.

Ribozyme and antisense antiviral agents that are suitable for use in a subject combination therapy include, but are not limited to, ISIS 14803 (ISIS Pharmaceuticals/Elan Corporation; see, e.g., Witherell (2001) Curr Opin Investig Drugs. 2(11):1523-9); Heptazyme™; and the like.

Any means for the introduction of such polynucleotides into mammals, human or non-human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat. Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxvirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, a subject therapy further comprises administering a palliative agent (e.g., an agent that reduces patient symptoms caused by the alphaviral infection), or other agent for the avoidance, treatment, or reduction of a side effect of a therapeutic agent. Such agents are also referred to as “symptom management agents” or “side effect management agents.” Suitable symptom management agents include agents that reduce one or more patient symptoms caused by the alphaviral infection. Suitable side effect management agents include agents for the avoidance, treatment, or reduction of a side effect of an agent that inhibits enzymatic activity of a cyclophilin; agents for the avoidance, treatment, or reduction of a side effect of a therapeutic agent.

Suitable symptom and/or side effect management agents include agents that are effective in pain management; agents that ameliorate gastrointestinal discomfort; analgesics, anti-inflammatories, antipsychotics, antineurotics, anxiolytics, and hematopoietic agents. In addition, the invention contemplates the use of any compound for palliative care of subjects suffering from pain or any other side effect in the course of treatment with a subject therapy. Exemplary palliative agents include acetaminophen, ibuprofen, and other NSAIDs, H2 blockers, and antacids.

Analgesics that can be used to alleviate pain in the methods of the invention include non-narcotic analgesics such as non-steroidal anti-inflammatory drugs (NSAIDs) acetaminophen, salicylate, acetyl-salicylic acid (aspirin, diflunisal), ibuprofen, Motrin, Naprosyn, Nalfon, and Trilisate, indomethacin, glucametacine, acemetacin, sulindac, naproxen, piroxicam, diclofenac, benoxaprofen, ketoprofen, oxaprozin, etodolac, ketorolac tromethamine, ketorolac, nabumetone, and the like, and mixtures of two or more of the foregoing.

Other suitable analgesics include fentanyl, buprenorphine, codeine sulfate, morphine hydrochloride, codeine, hydromorphone (Dilaudid), levorphanol (Levo-Dromoran), methadone (Dolophine), morphine, oxycodone (in Percodan), and oxymorphone (Numorphan). Also suitable for use are benzodiazepines including, but not limited to, flurazepam (Dalmane), diazepam (Valium), and Versed, and the like.

Suitable anti-inflammatory agents include, but are not limited to, steroidal anti-inflammatory agents, and non-steroidal anti-inflammatory agents. For example, suitable steroidal anti-inflammatory agents include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures of two or more of the foregoing. In other embodiments, suitable non-steroidal anti-inflammatory agents, include, but are not limited to, 1) the oxicams, such as piroxicam, isoxicam, tenoxicam, and sudoxicam; 2) the salicylates, such as aspirin, disalcid, benorylate, trilisate, safapryn, solprin, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, al:minoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

Other suitable anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin. Sodium; Triclonide; Triflumidate; Zidometacin; and Zomepirac Sodium.

Antipsychotic and antineurotic drugs that can be used to alleviate psychiatric side effects in the methods of the invention include any and all selective serotonin receptor inhibitors (SSRIs) and other anti-depressants, anxiolytics (e.g. alprazolam), etc. Anti-depressants include, but are not limited to, serotonin reuptake inhibitors such as Celexa®, Desyrel®, Effexor®, Luvox®, Prozac®, Zoloft®, and Serzone®; tricyclics such as Adapin®, Anafrinil®, Elavil®, Janimmine®, Ludiomil®, Pamelor®, Tofranil®, Vivactil®, Sinequan®, and Surmontil®; monoamine oxidase inhibitors such as Eldepryl®, Marplan®, Nardil®, and Parnate®. Anti-anxiety agents include, but are not limited to, azaspirones such as BuSpar®, benzodiazepines such as Ativan®, Librium®, Tranxene®, Centrax®, Klonopin®, Paxipam®, Serax®, Valium®, and Xanax®; and beta-blockers such as Inderal® and Tenormin®.

Agents that reduce gastrointestinal discomfort such as nausea, diarrhea, gastrointestinal cramping, and the like are suitable palliative agents for use in a subject combination therapy. Suitable agents include, but are not limited to, antiemetics, anti-diarrheal agents, H2 blockers, antacids, and the like.

Suitable H2 blockers (histamine type 2 receptor antagonists) that are suitable for use as a palliative agent in a subject therapy include, but are not limited to, Cimetidine (e.g., Tagamet, Peptol, Nu-cimet, apo-cimetidine, non-cimetidine); Ranitidine (e.g., Zantac, Nu-ranit, Novo-randine, and apo-ranitidine); and Famotidine (Pepcid, Apo-Famotidine, and Novo-Famotidine).

Suitable antacids include, but are not limited to, aluminum and magnesium hydroxide (Maalox®, Mylanta®); aluminum carbonate gel (Basajel®); aluminum hydroxide (Amphojel®, AlternaGEL®); calcium carbonate (Tums®, Titralac®); magnesium hydroxide; and sodium bicarbonate.

Antiemetics include, but are not limited to, 5-hydroxytryptophan-3 (5HT3) inhibitors; corticosteroids such as dexamethasone and methylprednisolone; Marinol® (dronabinol); prochlorperazine; benzodiazepines; promethazine; and metoclopramide cisapride; Alosetron Hydrochloride; Batanopride Hydrochloride; Bemesetron; Benzquinamide; Chlorpromazine; Chlorpromazine Hydrochloride; Clebopride; Cyclizine Hydrochloride; Dimenhydrinate; Diphenidol; Diphenidol Hydrochloride; Diphenidol Pamoate; Dolasetron Mesylate; Domperidone; Dronabinol; Fludorex; Flumeridone; Galdansetron Hydrochloride; Granisetron; Granisetron Hydrochloride; Lurosetron Mesylate; Meclizine Hydrochloride; Metoclopramide Hydrochloride; Metopimazine; Ondansetron Hydrochloride; Pancopride; Prochlorperazine; Prochlorperazine Edisylate; Prochlorperazine Maleate; Promethazine Hydrochloride; Thiethylperazine; Thiethylperazine Malate; Thiethylperazine Maleate; Trimethobenzamide Hydrochloride; Zacopride Hydrochloride.

Anti-diarrheal agents include, but are not limited to, Rolgamidine, Diphenoxylate hydrochloride (Lomotil), Metronidazole (Flagyl), Methylprednisolone (Medrol), Sulfasalazine (Azulfidine), and the like.

Suitable hematopoietic agents that can be used to prevent or restore depressed blood cell populations in the methods of the invention include erythropoietins, such as EPOGEN™ epoetin-alfa, granulocyte colony stimulating factors (G-CSFs), such as NEUPOGEN™ filgrastim, granulocyte-macrophage colony stimulating factors (GM-CSFs), thrombopoietins, etc.

### Pharmaceutical Formulations

An active agent (e.g., a cyclophilin inhibitor, at least one additional therapeutic agent, etc.) can be administered to subjects in a formulation with a pharmaceutically acceptable excipient(s). The terms “active agent” and “therapeutic agent” are used interchangeably herein. A wide variety of pharmaceutically acceptable excipients are known in the art and have been. well described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent (e.g., a cyclophilin inhibitor, at least one additional therapeutic agent, etc.) may be administered to the host using any convenient means capable of resulting in the desired therapeutic effect. Thus, an active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent can be achieved in various ways, including oral, buccal, rectal, parenteral, intraperitoneal, intradermal, subcutaneous, intramuscular, transdermal, intratracheal, etc., administration. In some embodiments, two different routes of administration can be used.

Subcutaneous administration of an active agent (e.g., a cyclophilin inhibitor, at least one additional therapeutic agent, etc.) can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system. Such delivery methods may be especially useful for administration to cell types in the CNS.

In some embodiments, an active agent (e.g., a cyclophilin inhibitor, at least one additional therapeutic agent, etc.) is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art. Such delivery methods may be especially useful for administration to cell types in the CNS.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned, such as a site within the CNS. Implantation sites include, but are not necessarily limited to a subdermal, subcutaneous, intramuscular, or other suitable site within a subject's body. Subcutaneous implantation sites are generally used because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump are also suitable for use with the present invention. Examples of such devices include those described in, for example, U.S. Pat. Nos. 4,692,147; 4,360,019; 4,487,603; 4,360,019; 4,725,852, and the like. In general, a subject treatment method can be carried out using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)).

Exemplary osmotically-driven devices suitable for use in a subject treatment method include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted above, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent (e.g., a cyclophilin inhibitor, at least one additional therapeutic agent, etc.) is formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent (e.g., a cyclophilin inhibitor, at least one additional therapeutic agent, etc.) calculated in an amount sufficient to produce the desired effect in association with a pharmaceutically acceptable diluent, carrier or vehicle. The specifications for the unit dosage forms depend on the particular agent employed and the effect to be achieved, and the pharmacodynamics associated with each agent in the host.

In some embodiments, an active agent (e.g., a cyclophilin inhibitor, at least one additional therapeutic agent, etc.) is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

The amount of active ingredient (e.g., an agent that inhibits one or more functions, such as enzymatic activity, of a cyclophilin) that may be combined with carrier materials to produce a dosage form can vary depending on the host to be treated and the particular mode of administration. A typical pharmaceutical preparation can contain from about 5% to about 95% active ingredient (w/w), and in some cases from about 95% to about 98%, or from about 98% to about 99% (w/w) active ingredient). In other embodiments, the pharmaceutical preparation can contain from about 20% to about 80% active ingredient.

Those of skill will readily appreciate that dose levels can vary as a function of the specific agent that inhibits one or more functions (e.g., enzymatic activity) of a cyclophilin, the severity of the symptoms and the susceptibility of the subject to side effects. Preferred dosages for a given agent that inhibits enzymatic activity of a cyclophilin are readily determinable by those of skill in the art by a variety of means. A typical means is to measure the physiological potency of a given active agent.

In some embodiments, multiple doses of an agent that inhibits enzymatic activity of a cyclophilin are administered. For example, an agent that inhibits enzymatic activity of a cyclophilin is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments of combination therapies, the additional antiviral agent(s) can be administered during the entire course of treatment with a cyclophilin inhibitor, and the beginning and end of the treatment periods coincide. In other embodiments, the additional antiviral agent(s) is administered for a period of time that is overlapping with that of the cyclophilin inhibitor treatment, e.g., treatment with the additional antiviral agent(s) begins before the cyclophilin inhibitor treatment begins and ends before the cyclophilin inhibitor treatment ends; treatment with the additional antiviral agent(s) begins after the cyclophilin inhibitor treatment begins and ends after the cyclophilin inhibitor treatment ends; treatment with the additional antiviral agent(s) begins after the cyclophilin inhibitor treatment begins and ends before the cyclophilin inhibitor treatment ends; or treatment with the additional antiviral agent(s) begins before the cyclophilin inhibitor treatment begins and ends after the cyclophilin inhibitor treatment ends.

The cyclophilin inhibitor can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional antiviral agents.

### EXEMPLIFICATION

This invention is further illustrated by the following examples, which should not be construed as limiting.

### Example 1

### Materials and Methods for Examples 2-4

### A. Viruses

Two strains of Sindbis virus were used in the microarray analyses described below: TE and Double Mutant (SINV DM). TE is a recombinant strain of SINV adapted for increased neurovirulence by the presence of His-55 in the E2 protein (Levine and Griffin (1993) J. Virol. 67:6872-6875 and Ubol et al. (1994) Proc. Natl. Acad. Sci. USA 91:5202-5206). Sindbis virus possesses four nonstructural proteins that play important roles in virus replication. The DM strain was generated in a previous study by changing asparagines at position 10 and 24 of the macro domain of nonstructural protein 3 (nSP3) into alanines (Park and Griffin (2009) Virol. 388:305-314). DM SINV has impaired replication and amplification compared to TE SINV in mature neurons, although the precise reasons for this and the function of nSP3 are unclear.

### B. Cell Culture

The rat CSM14.1 nigral neuronal cell line, immortalized with the temperature-sensitive simian virus 40 T antigen, was a gift from Dale E. Bredesen (Buck Institute for Age Research, Novato, Calif.). Cells were grown at the permissive temperature of 31° C. in Dulbecco's modified Eagle's medium (DMEM; Gibco BRL, Grand Island, N.Y.) supplemented with 10% heat-inactivated fetal bovine serum (FBS), 100 U of penicillin/ml, 100 μg of streptomycin/ml, and 2 mM glutamine (Gibco BRL). Cycling CSM14.1 cells (cCSM) grown to 95% confluency in 6 well plates, were differentiated by shifting to the restrictive temperature 39° C., and replacing the growth medium with DMEM/1% FBS. Cells were cultured in these conditions for 3 weeks to achieve a differentiated state (dCSM).

### C. Infection

CSM14.1 cells were plated onto 6-well plates and differentiated for 3 weeks in DMEM/1% FBS as described above. Live cells were harvested and counted by tryptan blue exclusion. Stock TE SINV titered to 3×10̂9 pfu/mL was diluted in DMEM/FBS 1% to provide a multiplicity of infection (MOI) of 10. Cells were incubated for one hour with virus-containing media at 39° C. and rocked every 15 minutes to ensure an even distribution of virus. This media was aspirated and replaced with DMEM/1% FBS.

### D. Drug Treatment

Differentiated CSM14.1 cells were treated with nuclear factor of activated T-cells (NFAT)-blocking drugs Cyclosporin A and FK506 (Invitrogen) and the phosphodiesterase-inhibitor zaprinast (Invitrogen) suspended in dimethyl sulfoxide (DMSO). Following infection with SINV or mock-infection, virus-containing media was replaced with DMEM/1% FBS containing 10 μM Cyclosporin A, FK506, or zaprinast.

### E. Affymetrix Microarray Setup and Analyses

Differentiated CSM14.1 cells were mock-infected or infected with the TE strain of SINV as described above and harvested in triplicate at 3, 24, and 48 hours post infection. RNA extraction was performed using the RNAqueous Micro kit using the manufacturer's protocol (Ambion). RNA was quantified using a NanoDrop spectrophotometer, and quality assessed by RNA Nano LabChip analysis on an Agilent Bioanalyzer 2100. RNAs were hybridized to rat Affymetrix GeneChip microarrays ST1.0 using the WT-Ovation Pico RNA Amplification system version 1, WT-Ovation Exon Module version 1, and the FL-Ovation cDNA Biotin Module version 2 (NuGEN Technologies, Inc). For each sample, 20 nanograms of total RNA was amplified in a three-step process using Ribo-SPIA technology.

Washing and staining of eukaryotic targets using the signal amplification protocol were performed in an automated fluidics station (Affymetrix FS450) by Affymetrix protocol FS450_{—}0001. Arrays were scanned on a GCS3000 laser scanner with autoloader and 3G upgrade (Affymetrix) at an emission wavelength of 560 nm at 2.5 nm resolution. Quality assessment was executed using Expression console software (Affymetrix) by recommendations included in the white paper “QC metrics for Exon and Gene Design Expression Arrays” (Affymetrix).

Microarray data were then uploaded into Partek Genomics Suite (Partek Genomics Suite 6.2, Partek Incorporated, St. Louis, Mo., USA.) as CEL files. Raw data were processed using the GC-RMA algorithm and the batch effect of sample was removed. A principle component analysis (PCA) based on covariance was performed across samples and a 3-way Analysis of Variance (ANOVA) was performed based on infection type, timepoint, and a type*timepoint interaction. Genes were identified as differently expressed using a p<0.005 and FDR<0.05 cut off between mock-infected and virus-infected at each timepoint. Differentially expressed genes were organized into gene lists and compared by Venn diagram.

Lists of differentially-expressed genes generated through Partek Genomic Suite were uploaded into Ingenuity Pathway Analysis software (IPA, Ingenuity Systems, Mountain View, Calif.) for further analysis. IPA compares data to a large curated database of network-categorized protein relationships taken from the literature. When analyzing a gene list, IPA calculates a score for each database network according to its fit. This score is the negative base-10 logarithm of the P value indicating the likelihood of compared proteins in a hypothetical network being found together by random chance (Raponi et al. (2004) BMC Cancer 4:56). Connected networks have associated canonical pathways and functions that are similarly scored. For each gene list, canonical pathways, especially those related to neurological disease and immune response, were explored for patterns.

### F. Reverse-Transcriptase Polymerase Chain Reaction

To confirm the microarray analysis, reverse transcriptase polymerase chain reaction was performed for several mRNAs identified as differently-expressed and key to pathways of interest from Ingenuity Pathway Analysis. Differentiated CSM 14.1 cells were mock-infected or SINV-infected as described above. At 3, 24, and 48 hours post infection, dCSM14.1 cells were washed with phosphate-buffered saline (PBS), trypsinized, and centrifuged for 1 minute at 12,000 rpm. Samples were washed with ice-cold PBS, centrifuged again, and stored at −80° C. Total RNA was purified from samples using the Qiagen RNease® Mini Kit (Qiagen). Each cDNA was synthesized from RNA using the SuperScript®III One-Step RT-PCR with Platinum® Taq kit (Invitrogen) and primer sets from Table 2. Reactions were run on a 2720 Thermal Cycler (Applied Biosystems). PCR products were loaded with DNA loading buffer on a 1% agarose gel and run at 100V for 1 hour. Gels were photographed using a Biorad Gel Doc XR System (Biorad).

TABLE 2

| | Forward | Reverse | |
|---|---|---|---|
| Gene | Primer | Primer | Provider |
| CALM1 | 5′- ATC CGT | 5′- TGA TGG | Integrated |
| | GAG GCA TTC | TGT GCT CAA | DNA |
| | CGA GTC TTT - | GTC CAC AGA - | Technologies |
| | 3′ | 3′ | |
| CALM2 | 5′- TCG AGT | 5′- TCC ACA | Integrated |
| | GGT TGT CTG | CTT CAA CAG | DNA |
| | TTC TGG TCT - | CAA CAT GCC - | Technologies |
| | 3′ | 3′ | |
| CALM3 | 5′- AGA TGG | 5′- AGT GTT | Integrated |
| | CAC CAT TAC | GAA GAG AGG | DNA |
| | CAC CAA GGA - | AGA GCG CAA - | Technologies |
| | 3′ | 3′ | |
| ITPR3 | 5′- ATG AAC | 5′- AAT GTT | Integrated |
| | ACA CCG TGG | TGA AGA GCA | DNA |
| | TGA TGG AGA - | GCT GCA GGG - | Technologies |
| | 3′ | 3′ | |
| CACNA1A | 5′- ATC ACC | 5′- AAC CAG | Integrated |
| | ATG GAA GGC | GTT TCT GAG | DNA |
| | TGG ACT GAT - | AGA TGC CCA - | Technologies |
| | 3′ | 3′ | |
| CACNA1D | 5′- TAC AAG | 5′- AGC TGC | Integrated |
| | CAC GAC CGA | CTG GGT ACC | DNA |
| | GAG CTT CAA - | TGT TGT AAT - | Technologies |
| | 3′ | 3′ | |
| CACNA1E | 5′- TAA AGC | 5′- TGT CCT | Integrated |
| | TTC TGC GAC | CAG CCA CTG | DNA |
| | AGG GCT ACA - | GCA TAT TCA - | Technologies |
| | 3′ | 3′ | |
| CACNA1C | 5′- TAG CCT | 5′- AAA CGT | Integrated |
| | GTA TGC CAG | CTG AGG GAA | DNA |
| | GCA AGA AGT - | GTT GCT CCT - | Technologies |
| | 3′ | 3′ | |
| PDE5A | 5′- ACA AAG | 5′- AGC TGG | Integrated |
| | GCA TTG TGG | GCG TTG TGA | DNA |
| | GAC ATG TGG - | AGA ACA ATG - | Technologies |
| | 3′ | 3′ | |
| B-actin | 5′- AGC CAT | 5′- CTC TCA | Integrated |
| | GTA CGT AGC | GCT GTG GTG | DNA |
| | CAT CC -3′ | GTG AA -3′ | Technologies |

### G. Subcellular Fractionation

Differentiated CSM 14.1 cells were mock infected or SINV-infected as described above. At 3, 24, and 48 h post treatment, dCSM14.1 cells were washed with ice-cold phosphate-buffered saline (PBS) and lysed with 50 μL Lysis B (0.25 M Sucrose, 0.1 mM EGTA, 10 mM Tris, 1× protease inhibitor, 1× phosphatase inhibitor, pH 7.5) buffer. Samples were sonicated for 30 pulses with an output of 40 and a duty cycle of 2.5 using a VW Scientific Sonifier 250 then centrifuged at 10,000 RPM for 1 hour at 4° C. The supernatant (cytoplasmic fraction) was removed and the pellet (nuclear fraction) was resuspended in Lysis B solution.

### H. Western Blot Analysis

Ten μg of protein was taken from each sample and denatured by boiling for 5 minutes in 6×SDS loading buffer (0.5 M Tris [pH 6.8] 30% glycerol, 10% SDS, 0.12% bromophenol blue, 6% β-mercaptoethanol), followed by 4-20% Tris gel electrophoresis (NuPage). For immunoblotting, proteins were transferred to nitrocellulose membranes and blocked with 5% skim milk in Tris-buffered saline containing 0.2% Tween-20. Membranes were probed with monoclonal antibodies at concentrations listed in Table 3.

TABLE 3

| Antibody | Provider | Catalog # | Dilution |
|---|---|---|---|
| NFATc1 | Santa Cruz | Sc-13033 | 1:10000 in. Primary |
| | Biotechnology, Inc. | | Antibody Dilution |
| | | | Buffer |
| Phospho-NFATc1 | Santa Cruz | Sc-32979 | 1:10000 in Primary |
| | Biotechnology, Inc. | | Antibody Dilution |
| | | | Buffer |
| NFAT3 Rabbit | Cell-Signaling | 2183 | 1:10000 in Primary |
| mAb | Technology | | Antibody Dilution |
| | | | Buffer |
| Histone H3Rabbit | Cell-Signaling | 9717 | 1:2000 in Primary |
| mAb | Technology | | Antibody Dilution |
| | | | Buffer |
| GAPDH Rabbit | Cell-Signaling | 2118 | 1:2000 in Primary |
| mAb | Technology | | Antibody Dilution |
| | | | Buffer |
| Mouse Anti-Actin | Chemicon-Millipore | MAB1501 | 1:10000 in Primary |
| Monoclonal Ab | | | Antibody Dilution |
| | | | Buffer |

### I. Cell Viability Count

Differentiated CSM 14.1 cells were divided into 5 groups: mock-infected, SINV-infected, SINV-infected+cyclosporin A, SINV-infected+FK506, and SINV-infected+zaprinast. At 3, 24, and 48 h post infection/treatment, cells were trypsinized and resuspended in 50 μL DMEM supplemented with 1% FBS. Cells were then stained with 1% Trypan Blue and counted in a hemocytometer. Counts were performed blind and in triplicate.

### J. Plaque Assay

Sampled supernatant fluids from differentiated CSM14.1 cells organized into different treatment groups described above were taken at 0, 6, 12, 24, 36, 48 and 60 hours post-infection and stored at −80° C. These supernatant fluids were serially-diluted in DMEM supplemented with 1% FBS and used for virus titration by infecting monolayers of BHK cells in 6 well plates. After 48 hours, cells were stained with Neutral Red. Plaques were counted to calculate plaque—forming units/mL.

### Example 2

### Identification of Genes Differentially Expressed Upon Alphaviral Infection

SINV infection induces rapid death in immature, cycling neurons but not differentiated neurons. The nsP3 double mutant strain of SINV similarly induces greater mortality in cycling than differentiated neurons, but has reduced replication (Park and Griffin (2009) Virol. 388:305-314). CSM14.1 is a rat nigral cell line that differentiates under restrictive culture conditions. To analyze differences in mRNA expression, differentiated CSM14.1 cells that were mock-infected (M), wild type SINV-infected (WT), or double mutant SINV-infected (DM) were harvested at specified times and submitted for microarray processing.

Genome-wide differences in mRNA expression were visualized by Principal Component Analysis, a tool which reduces complex, multidimensional microarray datasets to three dimensions. At all time points, DM samples were very similar to M samples. WT virus samples showed substantial differences from both DM and M at 24 hours post-infection and differences increased at 48 hours post-infection. WT samples also grew distant from each other at 24 and 48 hours post-infection, indicating that viral effects on transcription are broadly variable.

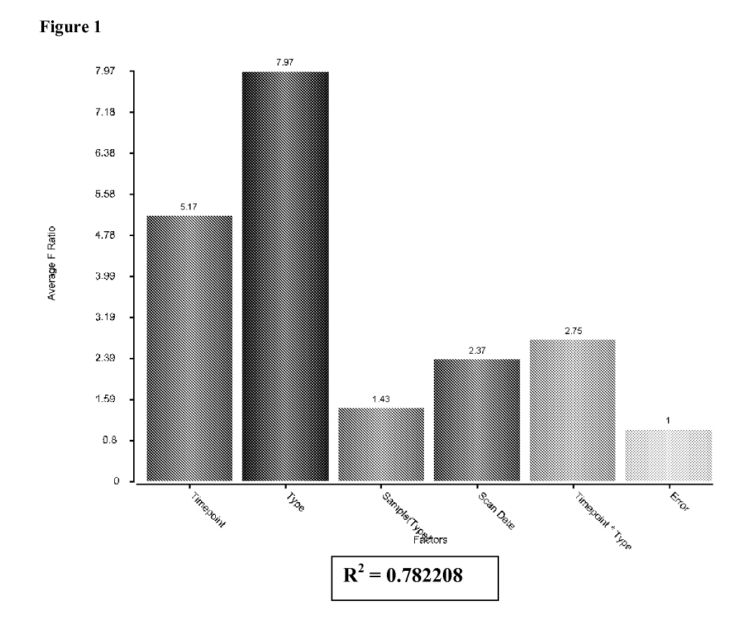

To assess the impact of individual factors on the variance observed in samples, a source of variation analysis was run on the microarray data (FIG. 1). In this analysis, F ratio represents the signal-to-noise ratio for variance within the ANOVA model. The average R² value for all probe sets in the model was 0.782208. Type of infection played the largest role in accounting for variation with an F ratio of 7.97, followed by time point with an F ratio of 5.17. The interaction between time point and infection type had an F ratio of 2.75, and the batch effects of scan date and sample had ratios of 2.37 and 1.43 respectively, indicating that the variation observed was not an artifact of batch effects.

A 3-way Analysis of Variance (ANOVA) was run on the microarray data and lists of significantly differently-expressed probe sets were generated for the comparison of mock-infected and WT SINV-infected cells at each time point with a P value<0.005 and FDR<0.05. These lists were compared by Venn Diagram. The ST1.0 arrays used have a total of 80,555 probe sets. Different combinations of these probe sets represent each gene. The number of probe sets meeting the criteria for a significant difference in expression grew with each time point (9 at 3 hours, 1568 at 24 hours, 24760 at 48 hours).

To organize the genes into networks, gene lists were imported into Ingenuity Pathway Analysis (IPA). Ingenuity maps gene lists to a database of known molecular interactions and calculates a score for each possible network based on another randomly selected network having the same number of entries by chance. Networks are large sets of molecules that are related by direct and indirect interactions across a variety of pathways. At 48 hours, the top network for the comparison of mock infected to WT SINV infected neurons was “Cell Cycle, Cell-To-Cell Signaling and Interaction, Cellular Movement” (Network 1) with a score of 19, indicating a 1 in 10̂19 chance of another randomly selected network containing the same number of entries.

Within these networks, canonical pathways are smaller sets of molecules related by interaction that form a well-characterized pathway in the literature. At 48 hours, the top-scoring canonical pathway for the comparison of mock-infected to WT SINV-infected neurons was “Cellular Effects of Sildenafil” (Viagra) with a p-value of 3.27E-13 and a ratio of 44/113 differently-expressed genes. This pathway was followed in score ranking by Cardiac Hypertrophy Signaling (p-value 5.36E-13, ratio 63/202) and Cardiac B-adrenergic Signaling (6.64E-13, ratio 44/115). Within the canonical pathway Cellular Effects of Sildenafil, Phosphodiesterase 5A (PDE5A) is the primary drug target for inhibition (Cornblath et al. (1990) J. Neuroimmunol. 26:113-118). PDE5A controls the metabolism of cyclic GMP (cGMP) to 5′ GMP and its inhibition leads to a buildup of cGMP. Cyclic GMP activates PKG, which phosphorylates and regulates Ca2+ permeable transient receptor potential (TRP) channels and L-type calcium channels (Koitabashi et al. (2010) J. Mol. Cell. Cardiol. 48:713-724 and Hopkins et al. (2010) Antimicrob. Agents Chemo. 54:660-672). This acts as a negative feedback mechanism suppressing calcium influx when intracellular calcium is elevated, indicating that calcium regulation may play a role in infection.

The canonical pathway was overlaid with the gene lists for 24 and 48 hours. At 48 hours, infected cells have a 6.355 fold reduction of PDE5A compared to mock infected cells with a p-value of 9.142E-5. Other notable aspects of the pathway that are differentially regulated are an upregulation of the inositol 1,4,5-triphosphate receptor members ITPR3 (3.140 fold increase, p-value 3.205E-4) and ITPR2 (4.276 fold increase, p-value 3.695E-4), which are triggered by IP3 to release calcium from the endoplasmic reticulum into the cytoplasm (Cromptom (1999) Biochem J. 341:233-249). Concurrently, 5 transmembrane calcium channels were differentially regulated, of which 3 were upregulated during infection (ITPR3, CACN1D, and CACNA1E) and 2 were downregulated (CACNA1A and CACNA1C). These results suggested that regulation of cytoplasmic calcium is an important response to infection, prompting a search for relevant pathways tied to calcium regulation.

Nuclear Factor of Activated T cells (NFAT) refers to a family of 5 closely-related transcription factors that are critical for the regulated transcription of cytokine genes and other immune response genes. NFAT has also has been characterized as playing a key role in neuronal development (Graef et al. (2001) Cell 105:863-875). The 5 different members of the NFAT family all activate a closely-overlapping set of target genes and are all regulated by calcineurin, a calcium-dependant phophatase. Under normal conditions, NFAT is phosphorylated and remains in the cytoplasm. When dephosphorylated by calcineurin, NFAT is imported into the nucleus where it acts as a transcription factor (Macian (2005) Nat. Rev. Immunol. 5:472-484). This activation depends on a prolonged elevation of calcium in the cytoplasm facilitated by a release of endoplasmic reticulum stores of calcium followed by influx of extracellular calcium (Nguyen and DiGiovanni (2008) Int. J. Dev. Neurosci. 26:141-145).

Because of its close ties to calcium elevation, it was determined whether other proteins related to activation of NFAT were differentially regulated during infection.

Ingenuity's canonical pathway for “Role of NFAT in the Immune Response” was overlaid with the comparisons at 24 hours and 48 hours. Within this pathway, Ca²⁺ activates calmodulin (CALM), which activates the phosphatase calcineurin, which in turn dephosphorylates NFAT, activating it and triggering its import into the nucleus (Aramburu et al. (1998) Mol. Cell. 1:627-637). Calmodulin and members of the calcineurin family PPP3CC and PPP3R1 are upregulated during infection. Once activated, CK1, GSK3B and XPO1 all play roles in phosphorylation and export of NFAT, deactivating it. All are downregulated during infection, while AKT, an inhibitor of GSK3B, is upregulated. In addition, “Cardiac Hypertrophy Signaling” and “Cardiac B-Adrenergic Signaling” scored as the 2^{nd} and 3^{rd} canonical pathways, respectively, and each has been characterized as disease consequences of inappropriate, constitutive NFAT activation (Jeong et al. (2008) Circ. Res. 102:711-719 and Molkentin (2004) Cardiovasc. Res. 63:467-475).

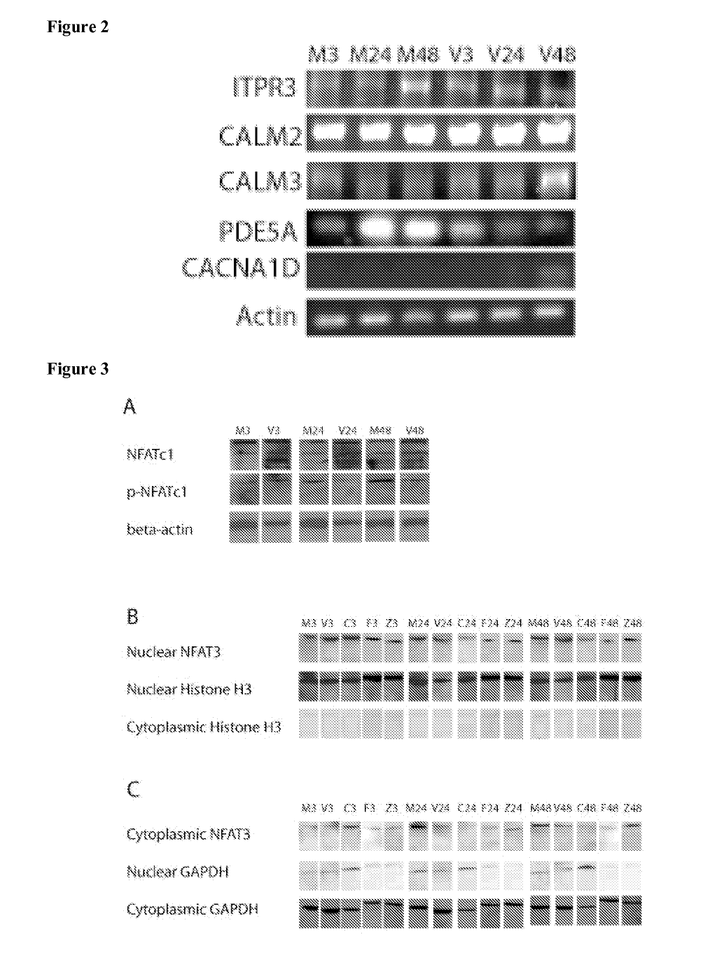

To confirm the data observed in microarray analysis, key molecules were selected on the basis of their assessed importance in the “Cellular Effects of Sildenafil” and “Role of NFAT in the Immune Response” pathways for reverse transcriptase PCR, including PDE5A, CALM2, CALM3, ITPR3 and CACNA1D. Each gene satisfied criteria as being significantly differently-expressed at 48 hours. PDE5A, CALM3, and CACNA1D had PCR products consistent with predictions based on the microarray data analyses (FIG. 2). CALM2 and ITPR3 did not show appreciable differences between samples (FIG. 2).

NFAT did not meet the cutoff for differential expression in the microarray analysis; however, previous studies have shown that the calcium-mediated pathway for NFAT activation regulates NFAT by protein interaction and not mRNA expression (Macian (2005) Nat. Rev. Immunol. 5:472-484 and Nguyen and DiGiovanni (2008) Int. J. Dev. Neurosci. 26:141-145). To examine the activation state of NFAT during viral infection, differentiated CSM14.1 cells were infected with TE SINV and Western blot analyses were performed at 3, 24, and 48 hours post-infection. Blotting with phospho-specific antibodies for NFATc1 showed that NFAT is dephosphorylated during viral infection (FIG. 3) and indicated that NFAT is activated during viral infection.

### Example 3

### Intracellular Localization of NFAT During Alphaviral Infection and Treatment with NFAT-Mediating Agents

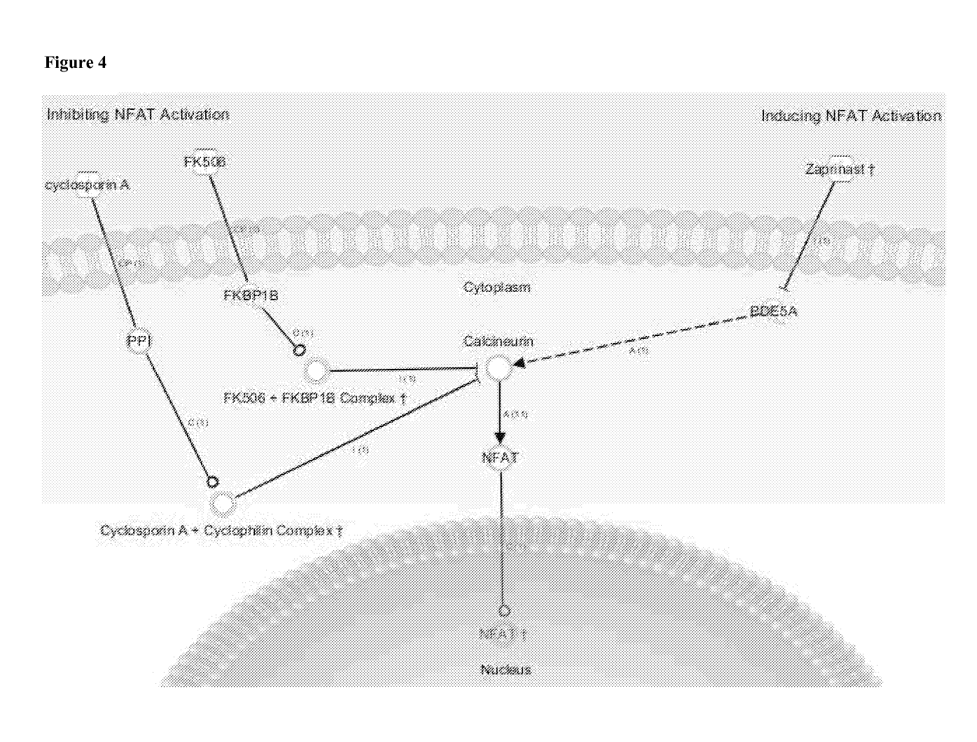

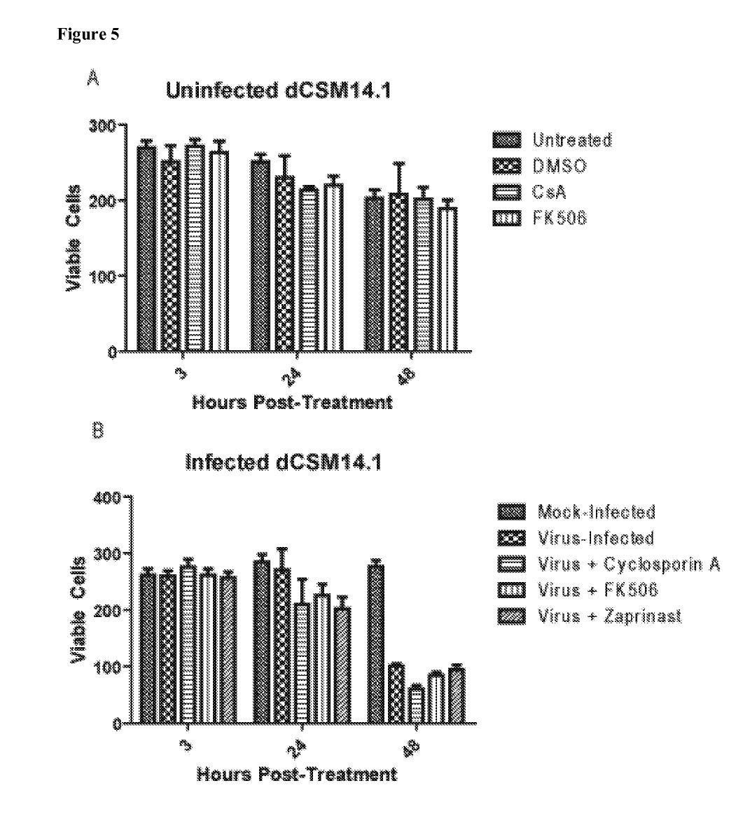

Activation of NFAT is dependent on calcineurin, a protein whose function is targeted by cyclosporin A and FK506 (FIG. 4). Cyclosporin A and FK506 bind to cyclophilin and FKBP12 (FK506 binding protein) respectively, each forming a complex that binds calcineurin, inhibits its activity and prevents the dephosphorylation and nuclear localization of NFAT. Zaprinast, similarly to sildenafil, inhibits phosphodiesterase 5 (FIG. 4). Given the connection of the sildenafil pathway with NFAT activation in the microarray data, it was determined whether treatment with a phosphodiesterase 5 inhibitor during viral infection would result in a more potent activation and nuclear localization of NFAT than viral infection alone.

To assess the intracellular localization of NFAT, differentiated CSM14.1 cells were infected with SINV for one hour and exposed to four conditions: mock-treatment, Cyclosporin A at 10 uM, FK506 at 10 uM, and zaprinast at 10 uM. The cells were then harvested at 3, 24, and 48 hours and separated into subcellular fractions. Western blot analyses were performed on nuclear and cytoplasmic fractions with an antibody specific only to NFAT3 (FIG. 3), which is another member of the NFAT family. The switch to analysis of NFAT3 was made due to probe availability and the identical conditions for regulation between members of the NFAT family. The untreated, SINV-infected group had increased nuclear NFAT3 and reduced cytoplasmic NFAT3 compared to uninfected cells at 24 and 48 hours post-infection.

The drug treatments had distinctly different outcomes. With Cyclosporin A treatment, both nuclear and cytoplasmic NFAT3 decreased at 24 hours and partially recovered at 48 hours post-infection compared to infected, untreated cells. FK506-treated cells showed the same pattern for nuclear NFAT3, but cytoplasmic NFAT3 remained constant. With zaprinast treatment, nuclear and cytoplasmic NFAT3 remained constant at all timepoints. These data indicated that Cyclosporin A had a strong suppressive effect on NFAT3 activation, FK506 had a weaker suppressive effect, and zaprinast had no effect.

### Example 4

### Effects of NFAT-Modulating Drugs on Cell Survival in Alphaviral-Infected Cells

To ascertain the impact on viability that NFAT-modulating drugs would have during the course of viral infection, infected cells were treated and viable cells counted at 3, 24, and 48 hours post SINV infection. Viability counts showed that cyclosporin A substantially increased mortality for infected cells (p<0.01), while FK506 and zaprinast did not detectably affect cell survival (FIG. 5B). Additionally, uninfected cells were treated with DMSO, cyclosporin A, and FK506 and viable cells counted at 3, 24, and 48 hours post-treatment. None of the treatments detectably reduced viability in uninfected cells (FIG. 5A).

### Example 4

### Effects of NFAT-Modulating Drugs on Alphaviral Replication in Alphaviral-Infected Cells

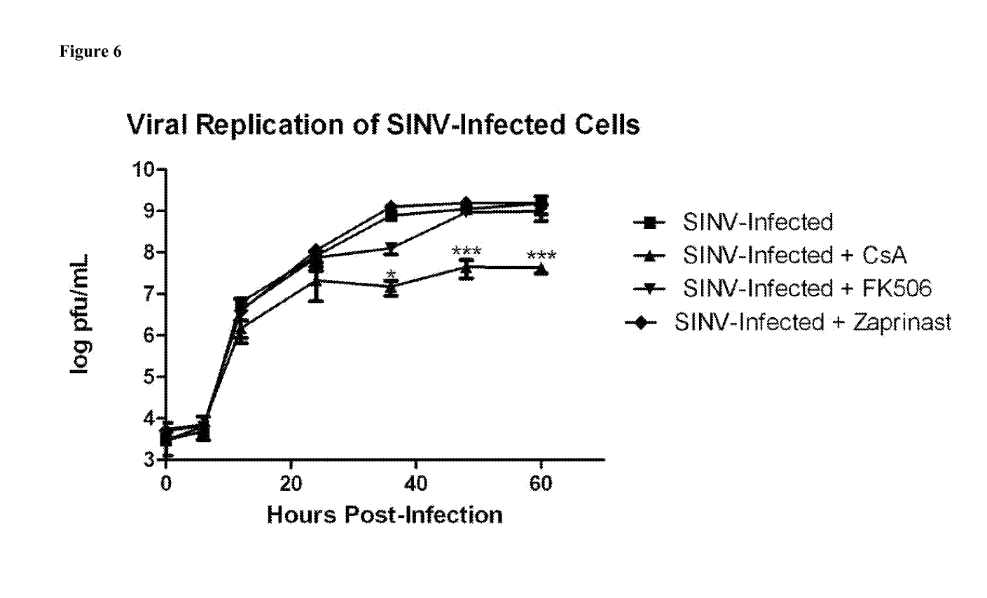

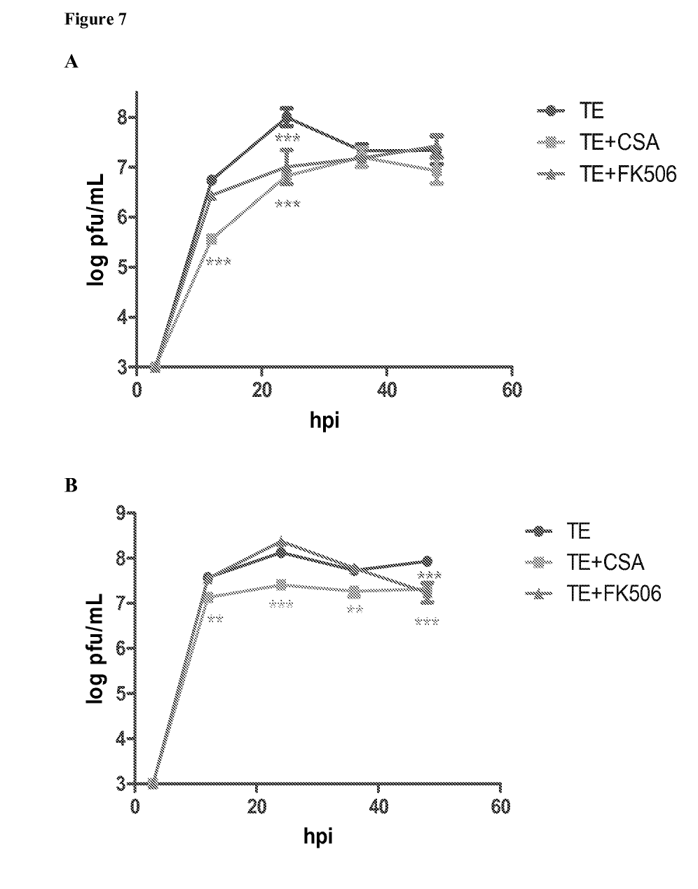

Differentiated CSM14.1 cells were infected for 1 hour and then treated with either cyclosporin A, FK506, or zaprinast at 10 uM concentration. Supernatant fluids were collected at 0, 6, 12, 24, 36, 48, and 60 hours post-infection. Viral titers were determined by plaque assay and expressed as plaque-forming units (PFU) per mL. Cyclosporin A treatment resulted in viral titers consistently 10-100 times lower than those for untreated SINV infected cells 24-60 hours after infection (FIG. 6). Although FK506 treatment resulted in lower titers than untreated SINV infection at all time points and zaprinast treatment tended to slightly increase viral titers at most time points, none of these differences were significant. Similar results were obtained using either undifferentiated or differentiated cells from another neuronal cell line, AP7 (FIG. 7).

Taken together, Cyclosporin A reduced survival of infected cells at 48 hours compared to untreated cells while FK506 had less of an effect. Interestingly, viral replication also was strongly reduced by treatment with cyclosporin A and only slightly reduced by FK506. These data indicate that reduced replication may be caused by premature apoptosis of infected cells. Cyclosporin A, by aggravating SINV-induced death, may reduce the virus's opportunity to replicate in addition to other possible direct antiviral effects.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of inhibiting alphavirus replication in a medium, comprising applying to said medium an effective amount of an agent that inhibits one or more functions of a cyclophilin.

2. The method of claim 1, wherein the alphavirus is selected from the group consisting of Aura virus, Babanki, Barmah Forest virus, Bebaru virus, Buggy Creek, Cabassou virus, Chikungunya virus, Eastern equine encephalitis virus, Everglades virus, Fort Morgan virus, Getah virus, Highlands J virus, Kyzylagach virus, Mayaro virus, Middelburg virus, Mosso das Pedras virus, Mucambo virus, Ndumu virus, O'nyong-nyong virus, Pixuna virus, Rio Negro virus, Ross River virus, Sagiyama virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, and Whataroa virus.

3. The method of claim 1, wherein the one or more functions of the cyclophilin is peptidyl prolyl isomerase enzymatic activity.

4. The method of claim 1, wherein the cyclophilin inhibitor inhibits the cyclophilin's peptidyl prolyl isomerase enzymatic activity and a) does not significantly inhibit calcineurin activity or b) does not significantly inhibit NFAT signaling.

5. (canceled)

6. The method of claim 1, wherein the agent comprises a cyclosporin, cyclosporin derivative, salts of a cyclosporin or cyclosporin derivative, or mixtures thereof.

7. The method of claim 6, wherein the cyclosporin is cyclosporin A, cyclosporin A derivative, salt of cyclosporin A or cyclosporin A derivative, or mixtures thereof.

8. The method of claim 1, wherein the cyclophilin is selected from the group consisting of cyclophilin A, B, C, D, E, F, G, H, I, J, K, L, M, N, O, P, Q, R, S, T, U, V, W, X, Y and Z.

9-11. (canceled)

12. The method of claim 1, wherein the alphavirus replication in the medium occurs in human cells.

13. A method of treating an alphavirus infection in a subject, comprising administering to the subject an effective amount of an agent that inhibits one or more functions of a cyclophilin.

14. The method of claim 13, wherein the alphavirus is selected from the group consisting of Aura virus, Babanki, Barmah Forest virus, Bebaru virus, Buggy Creek, Cabassou virus, Chikungunya virus, Eastern equine encephalitis virus, Everglades virus, Fort Morgan virus, Getah virus, Highlands J virus, Kyzylagach virus, Mayaro virus, Middelburg virus, Mosso das Pedras virus, Mucambo virus, Ndumu virus, O'nyong-nyong virus, Pixuna virus, Rio Negro virus, Ross River virus, Sagiyama virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, and Whataroa virus.

15. The method of claim 13, wherein the one or more functions of the cyclophilin is peptidyl prolyl isomerase enzymatic activity.

16. The method of claim 13, wherein the cyclophilin inhibitor inhibits the cyclophilin's peptidyl prolyl isomerase enzymatic activity and a) does not significantly inhibit calcineurin activity or b) does not significantly inhibit NFAT signaling.

17. (canceled)

18. The method of claim 13, wherein the agent comprises a cyclosporin, cyclosporin derivative, salts of a cyclosporin or cyclosporin derivative, or mixtures thereof.

19. The method of claim 18, wherein the cyclosporin is cyclosporin A, cyclosporin A derivative, salt of cyclosporin A or cyclosporin A derivative, or mixtures thereof.

20. The method of claim 13, wherein the cyclophilin is selected from the group consisting of cyclophilin A, B, C, D, E, F, G, H, I, J, K, L, M, N, O, P, Q, R, S, T, U, V, W, X, Y and Z.

21-23. (canceled)

24. The method of claim 13, further comprising administering to the subject an effective amount of at least one additional therapeutic agent.

25. The method of claim 24, wherein the at least one additional therapeutic agent reduces alphaviral replication, reduces the time to alphaviral clearance, reduces morbidity or mortality in the clinical course of the alphaviral infection, reduces subject symptoms caused by the alphaviral infection, or reduces a side effect of the cyclophilin inhibitor.

26. (canceled)

27. The method of claim 25, wherein the at least one additional therapeutic agent is selected from the group consisting of a nucleoside analog, mycophenolic acid, inhibitors of inosine monophosphate dehydrogenase (IMPDH), anti-alphaviral neutralizing monoclonal antibodies, poly ICLC, triaryl pyrazolin, anti-alphaviral ribozymes, zinc-finger antiviral protein, lactoferrin, and anti-alphaviral antisense RNA inhibitors.

28. The method of claim 24, wherein the cyclophilin inhibitor and at least one additional therapeutic agent are administered together as part of a single composition or wherein the cyclophilin inhibitor and at least one additional therapeutic agent are administered separately.

29. (canceled)

30. The method of claim 13, wherein the administering step comprises administering the agent to the central nervous system.

31. The method of claim 13, wherein the subject is a human.
